(19) United States
(12) Patent Application Publication
(10) Pub. No.: US 2005/0251267 A1
(43) Pub. Date: Nov. 10, 2005

(54) Cell permeable structural implant

(76) Inventors: John Winterbottom, Jackson, NJ (US); Ryan Belaney, Old Bridge, NJ (US); David Knaack, Summit, NJ (US); Todd Boyce, Matawan, NJ (US); Lawrence Shimp, Morganville, NJ (US); Samuel Lee, Red Bank, NJ (US); David Kaes, Toms River, NJ (US)

Correspondence Address: CHOATE, HALL & STEWART LLP, TWO INTERNATIONAL PLACE, BOSTON, MA, 02110, US

(21) Appl. No.: 11/047,992

(22) Filed: Jan. 31, 2005

Related U.S. Application Data
(60) Provisional application No. 60/568,472, filed on May 4, 2004.

(51) Int. Cl. A61F 2/28; A61F 2/02; C12N 5/08

(52) U.S. Cl. 623/23.63; 435/398; 623/23.76; 623/23.51

(57) ABSTRACT

An implant including a cell conducting phase and a binder phase. At least a portion of the surface of the implant includes the cell conducting phase, and the cell conducting phase defines a path from the surface of the implant to an interior of the implant.

This application claims priority to U.S. Provisional Application No. 60/568,472, the entire contents of which are incorporated herein by reference.

### FIELD OF THE INVENTION

This invention relates to a biocompatible composite, and, more specifically, to a biocompatible composite that has potential to develop a pathway for cell ingrowth that facilitates penetration of cells to the interior of the composite.

### BACKGROUND OF THE INVENTION

Bone is a composite material composed of hydroxyapatite, collagen, and a variety of noncollagenous proteins, as well as embedded and adherent cells. Bone can be processed into an implantable material, such as an allograft, for example, by treating it to remove the cells, leaving behind the extracellular matrix. The processed bone biomaterial can have a variety of properties, depending upon the specific processes and treatments applied to it, and may be combined with other biomaterials to form a composite that incorporates characteristics of both bone and the other biomaterials. For example, bone-derived materials may be processed into load-bearing mineralized grafts that support and integrate with the patient's bone or may alternatively be processed into soft, moldable or flowable demineralized bone biomaterials that have the ability to induce a cellular healing response.

The use of bone grafts and bone substitute materials in orthopedic medicine is well known. While bone wounds can regenerate, fractures and other orthopedic injuries take a substantial time to heal, during which the bone is unable to support physiologic loads. Metal pins, screws, plates, rods, and meshes are frequently required to replace the mechanical functions of injured bone. However, metal is significantly stiffer than bone. Use of metal implants may result in decreased bone density around the implant site due to stress shielding. Additionally, metal is less than ideal as an implant material because it remains at the healing site after healing has occurred and the need for the metal implant has passed.

Bone's cellular healing processes, through coordinated activity of osteoblast and osteoclast cells, permit cadaveric bone grafts and certain bone substitute materials to be removed and replaced by endogenous bone that is almost indistinguishable from the original. However, the use of cadaveric bone grafts is limited by the available shape and size of grafts and the desire to optimize both mechanical strength and replacement rate relative to the timeframe of fracture or defect healing at the skeletal site. Variations in bone size and shape among patients (and donors) also make monolithic bone grafts a less optimal substitute material. Some bone substitute materials and bone chips are quickly degraded but cannot immediately provide mechanical support. Cancellous bone allografts have open spaces for easy cellular penetration and biodegradation, but they lack appropriate initial strength for many load bearing applications. Cortical bone grafts are stronger than cancellous grafts but are more slowly and incompletely replaced by endogenous tissue. While the extent of integration of these grafts is generally considered adequate, endogenous replacement of the graft seldom exceeds more than 50% (Stevenson, et al., Factors affecting bone graft incorporation, Clin. Orthop. Rel. Res., 1996, 324:66-74; Burchardt, Biology of cortical bone graft incorporation, in Osteochondral Allografts, Friedlander, et al., eds., New York: Little and Brown, 1981, pp. 51-57).

Thus, it is desirable to have an orthopedic implant material that is load bearing and undergoes more extensive transformation into native tissue.

### DEFINITIONS

As used herein, “bioactive agents” is used to refer to compounds or entities that alter, inhibit, activate, or otherwise affect biological or chemical events. For example, bioactive agents may include, but are not limited to osteogenic, osteoinductive, and osteoconductive agents, anti-AIDS substances, anti-cancer substances, antibiotics, immunosuppressants (e.g., cyclosporine), anti-viral agents, enzyme inhibitors, neurotoxins, opioids, hypnotics, anti-histamines, lubricants, tranquilizers, anti-convulsants, muscle relaxants and anti-Parkinson agents, anti-spasmodics and muscle contractants including channel blockers, miotics and anti-cholinergics, anti-glaucoma compounds, anti-parasite, anti-protozoal, and/or anti-fungal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, targeting agents, neurotransmitters, proteins, cell response modifiers, and vaccines. In a certain preferred embodiments, the bioactive agent is a drug.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, the United States Pharmacopeia-25/National Formular-20, published by the United States Pharmcopeial Convention, Inc., Rockville Md., 2001, and the “Pharmazeutische Wirkstoffe”, edited by Von Keemann et al., Stuttgart/New York, 1987, all of which are incorporated herein by reference. Drugs for human use listed by the FDA under 21 C.F.R. §§330.5, 331 through 361, and 440 through 460 and drugs for veterinary use listed by the FDA under 21 C.F.R. §§500 through 589, all of which is incorporated herein by reference, are also considered acceptable for use in accordance with the present invention.

As used herein, “biodegradable”, “bioerodable”, or “resorbable” materials are materials that degrade under physiological conditions to form a product that can be metabolized or excreted without damage to organs. Biodegradable materials may be hydrolytically degradable, may require cellular and/or enzymatic action to fully degrade, or both. Other degradation mechanisms, e.g., thermal degradation due to body heat, are also envisioned. Biodegradable materials also include materials that are broken down within cells. Degradation may occur by hydrolysis, enzymatic degradation, phagocytosis, or other methods.

The term “biocompatible”, as used herein, is intended to describe materials that, upon administration in vivo, do not induce undesirable long term effects.

The term “biomolecules”, as used herein, refers to classes of molecules (e.g., proteins, amino acids, peptides, polynucleotides, nucleotides, carbohydrates, sugars, lipids, nucleoproteins, glycoproteins, lipoproteins, steroids, lipids, etc.) that are commonly found in cells and tissues, whether the molecules themselves are naturally-occurring or artificially created (e.g., by synthetic or recombinant methods). For example, biomolecules include, but are not limited to, enzymes, receptors, glycosaminoglycans, neurotransmitters, hormones, cytokines, cell response modifiers such as growth factors and chemotactic factors, antibodies, vaccines, haptens, toxins, interferons, ribozymes, anti-sense agents, plasmids, DNA, and RNA. Exemplary growth factors include but are not limited to bone morphogenic proteins (BMP's) and their active subunits. In some embodiments, the biomolecule is a growth factor, cytokine, extracellular matrix molecule or a fragment or derivative thereof, for example, a cell attachment sequence such as RGD.

The term “continuity,” as used herein, refers to the degree to which there is a path, connection, succession, or union from a portion of the cell conducting phase in a composite to another portion of the cell conducting phase and/or to a surface or surfaces of the composite. In some embodiments, continuity is provided because the individual particles of the cell conducting phase are contiguous or are close enough together that cells can easily migrate from one particle to another. Alternatively, or in addition, a cell conducting binder phase material may provide a path between cell conducting phase regions.

“Deorganified”, as herein applied to matrices, particles, etc., refers to bone or cartilage matrices, particles, etc., that were subjected to a process that removes at least part of their original organic content. In some embodiments, at least 1%, 10%, 20%, 30%, 40%, 50%, 60%, 70%, 80%, 90%, or 99% of the organic content of the starting material is removed.

“Nondemineralized”, as herein applied to bone particles, refers to bone particles that have not been subjected to a demineralization process (i.e., a procedure that totally or partially removes the original inorganic content of bone).

The term “osteoconductive”, as used herein, refers to the ability of a substance or material to provide surfaces which are receptive to the growth of new host bone.

“Osteoinductive”, as used herein, refers to the quality of being able to recruit cells from the host that have the potential to stimulate new bone formation. In general, osteoinductive materials are capable of inducing bone formation in soft tissue (e.g. muscle).

As used herein, the term “macroporosity” is used to describe porosity sufficiently large for cells to access the pores; e.g. having pores on the order of 100 μm in diameter or greater.

“Polynucleotide”, “nucleic acid”, or “oligonucleotide”: The terms “polynucleotide,” “nucleic acid,” or “oligonucleotide” refer to a polymer of nucleotides. The terms “polynucleotide”, “nucleic acid”, and “oligonucleotide”, may be used interchangeably. Typically, a polynucleotide comprises at least two nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thihymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyriboses, arabinose, and hexose), or modified phosphate groups (e.g., phosphorothioates and 5′-N-phosphoramidite linkages). The polymer may also be a short strand of nucleic acids such as siRNA.

“Polypeptide”, “peptide”, or “protein”: As used herein, a “polypeptide”, “peptide”, or “protein” includes a string of at least two amino acids linked together by peptide bonds. The terms “polypeptide, “peptide”, and “protein”, may be used interchangeably. Peptide may refer to an individual peptide or a collection of peptides. In some embodiments, peptides may contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In one embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “polysaccharide” or “oligosaccharide”, as used herein, refer to any polymer or oligomer of carbohydrate residues. The polymer or oligomer may consist of anywhere from two to hundreds to thousands of sugar units or more. “Oligosaccharide” generally refers to a relatively low molecular weight polymer, while “starch” typically refers to a higher molecular weight polymer. Polysaccharides may be purified from natural sources such as plants or may be synthesized de novo in the laboratory. Polysaccharides isolated from natural sources may be modified chemically to change their chemical or physical properties (e.g., phosphorylated, cross-linked). Carbohydrate polymers or oligomers may include natural sugars (e.g., glucose, fructose, galactose, mannose, arabinose, ribose, and xylose) and/or modified sugars (e.g., 2′-fluororibose, 2′-deoxyribose, and hexose). Polysaccharides may also be either straight or branch-chained. They may contain both natural and/or unnatural carbohydrate residues. The linkage between the residues may be the typical ether linkage found in nature or may be a linkage only available to synthetic chemists. Examples of polysaccharides include cellulose, maltin, maltose, starch, modified starch, dextran, and fructose. Glycosaminoglycans are also considered polysaccharides. Sugar alcohol, as used herein, refers to any polyol such as sorbitol, mannitol, xylitol, galactitol, erythritol, inositol, ribitol, dulcitol, adonitol, arabitol, dithioerythritol, dithiothreitol, glycerol, isomalt, and hydrogenated starch hydrolysates.

As used herein, the term “remodeling” describes the process by which native bone, processed bone allograft, whole bone sections employed as grafts, and other bony tissues are replaced with new cell-containing host bone tissue by the action of osteoclasts and osteoblasts. Remodeling also describes the process by which non-bony native tissue and tissue rafts are removed and replaced with new, cell-containing tissue in vivo.

“Small molecule”: As used herein, the term “small molecule” is used to refer to molecules, whether naturally-occurring or artificially created (e.g., via chemical synthesis), that have a relatively low molecular weight. Typically, small molecules have a molecular weight of less than about 5000 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body.

As utilized herein, the phrase “superficially demineralized” as applied to bone particles refers to bone particles possessing at least about 90 weight percent of their original inorganic mineral content. The phrase “partially demineralized” as applied to the bone particles refers to bone particles possessing from about 8 to about 90 weight percent of their original inorganic mineral content, and the phrase “fully demineralized” as applied to the bone particles refers to bone particles possessing less than about 8, for example, less than about 1, weight percent of their original inorganic mineral content. The unmodified term “demineralized” as applied to the bone particles is intended to cover any one or combination of the foregoing types of demineralized bone particles.

As used herein, the term “transformation” describes the process by which a material is removed from an implant site and replaced by host tissue after implantation. Transformation may be accomplished by combination of processes, including but not limited to remodeling, degradation, resorption, and tissue growth and/or formation. Removal of the material may be cell-mediated or accomplished through chemical processes, such as dissolution and hydrolysis.

### SUMMARY OF THE INVENTION

In one aspect, the invention is an implant including a cell conducting phase and a binder phase. At least a portion of the surface of the implant includes the cell conducting phase, and the cell conducting phase defines a path from the surface of the implant to an interior of the implant. At least a portion of the cell conducting phase, the binder phase, or both, may swell upon exposure to a physiological environment. The cell conducting phase may include particles having a distribution of aspect ratios, and volume fraction of the cell conducting phase may be at least as great as the percolation threshold of the implant for particles having an aspect ratio equal to the largest aspect ratio in the distribution.

The implant may provide an environment that, in vivo, allows cells and/or tissue ingrowth to penetrate into the implant at least 1 mm from the surface. At least a portion of the surface of the implant may include a cell conducting material. The cell conducting phase may include a connected cluster of cell conducting material that occupies at least 10%, 20%, 30%, 40%, 50%, 60%, 70%, 80%, or 90% of the area of a cross-section of the implant. The implant may lack porosity sufficiently large to permit the migration of cells.

The ratio of the cell conducting phase to the binder phase may exhibit a gradient proceeding from a portion of the surface of the implant to a pre-determined portion of an interior of the implant. The gradient may be in the direction of decreasing cell conductive phase to binder phase ratio. The cell conducting phase may define at least one blind path from a surface of the implant to a location in the interior of the implant. The cell conducting phase, the binder phase, or both, may include a bioactive agent, biomolecule, or small molecule. The concentration of any of these may exhibit a gradient between two pre-determined locations in the implant. The gradient may exhibit radial symmetry.

The cell conducting phase may include one or more of a tissue-derived material, an extracellular matrix component, a synthetic extracellular matrix analog, a polymer, and a ceramic material. The binder phase may include a cell conducting material and may include one or more of a polymer and an inorganic material. The implant may exhibit a gradient in its transformation rate, and a portion of the implant may be free of cell conducting material.

In another aspect, the invention is an implant comprising a cell conducting phase and a binder phase. At least one cross-section of the implant exhibits a connected cluster of the cell conducting phase that defines a path from the surface of the implant to a location in the interior of the implant, for example, at least 1, 2, 3, 4, or 5 mm from the surface of the implant. The implant may provide an environment that allows cells, tissue, or both to penetrate at least 10%, 20%, 30%, 40%, or a larger amount of a radius of the implant into the implant from the surface.

In another aspect, the invention is a composite material including a cell conducting phase including bone fibers. The long axis of the bone fibers corresponds to a long axis of a bone from which the bone fibers were derived. The composite material further includes a binder phase combined with the cell conducting phase. In another aspect, the invention is an implant disposed in a tissue site in vivo and including a composite. The composite includes a cell conducting phase and binder phase. The cell conducting phase includes a cell-free bone-derived material, and at least one living cell derived from a host in which the implant is disposed within in the implant.

In another aspect, the invention is an implant disposed in an in vivo tissue site and including a composite. The composite includes a cell conducting phase and a binder phase. The cell conducting phase includes a cell-free bone-derived material, and living tissue provides mechanical communication between an interior of the implant and tissue exterior to the implant.

### BRIEF DESCRIPTION OF THE DRAWING

The invention is described with reference to the several figures of the drawing, in which,

FIG. 1 is a schematic diagram of the use of a milling machine having a non-helical end mill to produce bone fibers.

FIG. 2 is a photograph of bone fibers produced from the milling machine depicted in FIG. 1.

FIGS. 3A and 3B illustrate the connectivity of particles in a system below (3A) and at (3B) the percolation threshold (Pc).

FIGS. 4A and B are Micro CT images of sections through composites produced with particle:polymer weight ratios of (A) 60:40 and (B) 70:30.

FIGS. 4C and D are computer enhanced images of FIGS. 4A and 4B, respectively, showing connected clusters of particles.

FIGS. 5A-C are Micro CT images of cross-sections through bone fiber-polymer composites having weight ratios of (A) 50:50, (B) 60:40, and (C) 70:30.

FIGS. 5D-F are computer enhanced versions of FIGS. 5A-C, respectively, indicating connected clusters of bone fibers within the composite.

FIG. 6 is a graph illustrating the relationship between cross-sectional shape and the percolation threshold (adapted from E. J. Garboczi, et al., Physical Review E, 52, 819-828, 1995).

FIG. 7 is a graph illustrating the average yield stress of bone fiber/DTE composites at different bone/polymer ratios (denoted by mass ratios, n=3).

FIG. 8 is a graph illustrating the variation in the average compressive yield stress of 70/30 bone/polymer composites containing different ratios of bone fibers and regularly dimensional bone particles (denoted by mass ratios, n=3).

FIG. 9. Wet compressive strength comparison among selected polymers. Dark Bars: Polymer only; Light bars: 25% polymer, 75% allograft bone by weight. PCL=Polycaprolactone; Tyrosine Polycarbonate=Poly (DTE Carbonate)

FIG. 10. Effects of bone particle size upon wet mechanical strength of the composite material. Polymer component is Poly DTE carbonate. Human bone, sieved to particle size ranges identified.

FIG. 11. Property degradation in vivo and in vitro for Poly DTE Carbonate/Bone composites, and polymer only. Squares: in vitro; Triangles: in vivo.

FIG. 12. a.) Healing implant in canine diaphyses at 28 days. Line indicates the boundary of the drill hole. b.) Central edge of healing implant in canine diaphyses at 26 weeks. Note the extensive cellularity and new bone bridging between particles. C=Existing cortical diaphyseal bone; N=New bone network forming within the implant; P=Particle of Canine allograft bone from the composite implant, devoid of osteocytes; T=Poly DTE tyrosine polymer, *=Regions of cellular activity.

FIG. 13. a.) Sectioned Micro CT image of canine diaphyseal implant of bone/polymer composite, and b.) after incorporation and bone bridging following 26 weeks healing (sectioned through implant cylinder). Implants from canine bone particles in poly DTE carbonate.

FIG. 14. Micro CT scan of a composite of bone and poly(lactide-co-glycolide) four weeks after implantation.

FIG. 15. Micro CT scan of a composite of bone and PolyDTE carbonate four weeks after implantation.

FIG. 16. Micro CT scan of a composite of bone and PolyDTE carbonate four weeks after implantation. The composite was abraded before implantation to expose the allograft.

FIG. 17 Light micrograph of a composite of bone and PolyDTE carbonate eight weeks after implantation, stained with Goldner's trichrome.

FIG. 18 is a graph describing the areas occupied by new bone (diamonds), allograft (squares), polymer (triangles) and cells/marrow (circles) in images of cross-sections of composite implants immediately, eight weeks, and twenty-four weeks following implantation.

### DETAILED DESCRIPTION OF CERTAIN PREFERRED EMBODIMENTS

The invention is a composite that includes structures which facilitate the development of pathways along which cells may migrate into the composite. The composite is essentially solid and may exhibit little significant porosity or macroporosity. Once implanted, a cell conducting phase in the composite facilitates cell ingrowth along certain paths, encouraging penetration of the cells to the interior of the material. Cells may penetrate the composite either directly through the cell conducting phase or at the interface of the cell conducting phase and the composite matrix (e.g., the bone/polymer interface). In one embodiment, the cell conducting phase swells upon exposure to a physiological environment, allowing cells to migrate within the cell conducting material. In another embodiment, the cell conducting phase gradually erodes or dissolves or is otherwise removed. Cells migrate to where the cell conducting phase is being removed and replace it with new endogenous tissue. Alternatively or in addition, cells make their own path through the material by degrading or transforming either the cell conducting phase or the composite matrix. Cells migrate along directions determined by the composition and microstructure of the composite, e.g., the location of the cell conducting phase. Alternatively or in addition, cells may degrade or transform a binder phase. Degradation may occur by hydrolysis, enzymatic degradation, phagocytosis, or other mechanisms. The composites need not provide porosity sufficiently large that cells can simply migrate into the composite. Rather, the composites provide a direction along which it is easier for cells to infiltrate the composite via the mechanisms described herein or some other mechanism. As the composite degrades, these cells synthesize new tissue to maintain the integrity of the material (both synthetic and endogenous) at the implant site. In this way, the mechanical properties of the site where the composite is implanted are maintained even as the composite degrades or is transformed. Regardless of the mechanism, cells behave as if the composite was porous and can penetrate deep into the composite.

In some embodiments, transformation occurs when penetration of a biomaterial having little or no macroporosity with living tissue precedes significant degradation of the structural integrity of the implant. In some embodiments, the implanted composite lacks porosity greater than 10 μm, 25 μm, 50 μm, or 75 μm. Thus, in the case of bone/polymer composites, significant resorption of the carrier polymer occurs after penetration of the implant by bone and vascular tissue. Transformation of the inventive biomaterials is distinguished from tissue growth or healing following implantation of conventional resorbable polymer or ceramic implants in that conventional polymer constructs may need to undergo significant resorption to provide a space for tissue ingrowth, or the resorbable construct may need to be fabricated with macroporosity to allow cell entry into the construct. Thus, tissue ingrowth into resorbable structures fabricated without significant porosity is limited by polymer resorption rates. Conversely, although tissue ingrowth may proceed quickly into macroporous resorbable polymer constructs, they may have compromised load bearing characteristics because of their inherent macroporosity.

The cell conducting phase may include bone fibers milled from whole bone or bone sections and oriented by the manufacturing process in such a manner that the long axis of the fibers is parallel to the long axis of the original bone. These fibers are incorporated into composites by combining them with a binder or matrix material at a volume fraction sufficient to provide a path from a surface of the composite to its interior along the bone fibers or successively connected bone fibers.

Preparation of a Cell Conducting Phase

The cell conducting phase facilitates migration of cells to the interior of the implant along paths defined by the cell conducting phase. As they migrate, some cells may replace the solid cell conducting material with new tissue, allowing the implant to interpenetrate with surrounding tissue. Other cells may find their way along the interface of the matrix/conducting phase by inserting themselves into the interface. The cell conducting phase may be osteoconductive, osteoinductive, or both. In one embodiment, the cell conducting material has tensile and shear strength greater than that of the binder phase and bonds fairly well with the binder phase. In this embodiment, the cell conducting phase may enhance the mechanical properties of the resulting composite.

Exemplary materials for use in the cell conducting phase include but are not limited to xenograft, allograft, or autograft tissues, including non-bony tissues, extracellular matrix, inorganic ceramics, synthetic polymers, and bone. Non-bony tissues suitable for use with the invention include connective tissue such as tendon, ligament, cartilage, endodermis, small intestinal mucosa, skin, hair, and muscle. The tissues may be excised and cut into a plurality of elongated fragments or particles employing methods known in the art. Reduction of the antigenicity of allogeneic and xenogeneic tissue can be achieved by treating the tissues with various chemical agents, e.g., extraction agents such as monoglycerides, diglycerides, triglycerides, dimethyl formamide, etc., as described, e.g., in U.S. Pat. No. 5,507,810, the contents of which are incorporated by reference herein. Small intestine submucosa tissue can be obtained and processed as described in U.S. Pat. No. 4,902,508, the contents of which are incorporated by reference herein. In summary, intestinal tissue is abraded to remove the outer layers, including both the tunica serosa and the tunica muscularis and the inner layers, including at least the luminal portion of the tunica mucosa. The resulting material is a whitish, translucent tube of tissue approximately 0.1 mm thick, typically consisting of the tunica submucosa with the attached lamina muscularis mucosa and stratum compactum. The tissue may be rinsed in 10% neomycin sulfate before use.

The implant may also be fabricated from either intact extracellular matrix or its components, alone or in combination, or modified or synthetic versions thereof. Exemplary extracellular matrix components include but are not limited to collagen, laminin, elastin, proteoglycans, reticulin, fibronectin, vitronectin, glycosaminoglycans, and other basement membrane components. Various types of collagen (e.g., collagen Type I, collagen Type II, collagen Type IV) are suitable for use with the invention. Collagens may be used in fiber, gel, or other forms. Sources for extracellular matrix components include, but are not limited to, skin, tendon, intestine and dura mater obtained from animals, transgenic animals and humans. Extracellular matrix components are also commercially available, for example, from Becton Dickenson. Collagenous tissue can also be obtained by genetically engineering microorganisms to express collagen as described, e.g., in U.S. Pat. No. 5,243,038, the entire contents of which are incorporated herein by reference. Procedures for obtaining and purifying collagen are well known in the art and typically involve acid or enzyme extraction as described, e.g., in U.S. Pat. No. 5,263,984, the contents of which are incorporated by reference herein. Exemplary synthetic ECM analogs include RGD-containing peptides, silk-elastin polymers produced by Protein Polymer Technologies (San Diego, Calif.) and BioSteel™, a recombinant spider silk produced by Nexia Biotechnologies (Vaudrevil-Dorion, QC, Canada). Various types of collagen (e.g., collagen Type I, collagen Type II, collagen Type IV) are suitable for use with the invention.

Ceramics and calcium phosphate materials may also be exploited for use with the invention. Exemplary inorganic ceramics for use with the invention include calcium carbonate, calcium sulfate, calcium phosphosilicate, sodium phosphate, calcium aluminate, calcium phosphate, hydroxyapatite, α-tricalcium phosphate, dicalcium phosphate, β-tricalcium phosphate, tetracalcium phosphate, amorphous calcium phosphate, octacalcium phosphate, and BIOGLASS™, a calcium phosphate silica glass available from U.S. Biomaterials Corporation. Substituted CaP phases are also contemplated for use with the invention, including but not limited to fluorapatite, chlorapatite, Mg-substituted tricalcium phosphate, and carbonate hydroxyapatite. Additional calcium phosphate phases suitable for use with the invention include those disclosed in U.S. Pat. Nos. RE 33,161 and RE 33,221 to Brown et al.; U.S. Pat. Nos. 4,880,610; 5,034,059; 5,047,031; 5,053,212; 5,129,905; 5,336,264; and 6,002,065 to Constantz et al.; U.S. Pat. Nos. 5,149,368; 5,262,166 and 5,462,722 to Liu et al.; U.S. Pat. Nos. 5,525,148 and 5,542,973 to Chow et al., U.S. Pat. Nos. 5,717,006 and 6,001,394 to Daculsi et al., U.S. Pat. No. 5,605,713 to Boltong et al., U.S. Pat. No. 5,650,176 to Lee et al., and U.S. Pat. No. 6,206,957 to Driessens et al, and biologically-derived or biomimetic materials such as those identified in Lowenstam H A, Weiner S, On Biomineralization, Oxford University Press, 234 pp. 1989, incorporated herein by reference.

Synthetic polymers may also be employed as a cell conductive phase. Exemplary polymers include, but are not limited to, tyrosine based polycarbonates and polyarylates such as those described by U.S. Pat. Nos. 5,587,507, 5,670,602, and 6,120,491, such as poly(desaminotyrosyltyrosine(ethyl ester)carbonate) (PolyDTE carbonate), poly(desaminotyrosyltyrosine carbonate) (PolyDT carbonate), and co-polymers of these in ratios of, e.g., 25:75, 40:60, 60:40, or 75:25. One skilled in the art will recognize that other osteoconductive polymers may also be used with the invention. Tyrosine-based polymers are naturally osteoconductive. Other polymers may be chemically modified, for example, by combination with calcium, calcium phosphates, hydroxyapatite, bioactive peptides and/or nucleic acids or other materials, for example, biomolecules, bioactive agents, and small molecules, to promote improved functionality or biological properties. For example, calcium ions may be chelated with unmodified or oxidized polymers to render them osteoconductive. Alternatively or in addition, natural or synthetic materials may be chemically modified to make them attractive to cells, for example, by derivatizing them with chemotactic factors or cell-adhesion sequences such as RGD.

In one embodiment, the bone particles are produced from fully mineralized human cortical bone. Bone particles for use in the composites of the invention may also be obtained from cortical, cancellous, and/or corticocancellous bone which may be of autogenous, allogenic and/or xenogeneic origin and may or may not contain cells and/or cellular components. Porcine and bovine bone are particularly advantageous types of xenogeneic bone tissue that may be used individually or in combination as sources for the bone particles. Bone particles for use in the composites of the invention may be any shape including, for example, irregular particulates, plates, fibers, helices and the like. Exemplary fibers may have a length between 0.05 and 500 mm, for example, between 5 and 100 mm, a thickness between 0.01 and 2 mm, for example, between 0.05 and 1 mm, and a width between 0.1 and 20 mm, for example, between 2 and 5 mm. As described herein, bone fibers are particles having at least one aspect ratio of 2:1 or greater. In some embodiments, bone fibers may have a ratio of width to length of at least 5:1, 10:1, 15:1, 25:1, 50:1, 200:1, or 500:1.

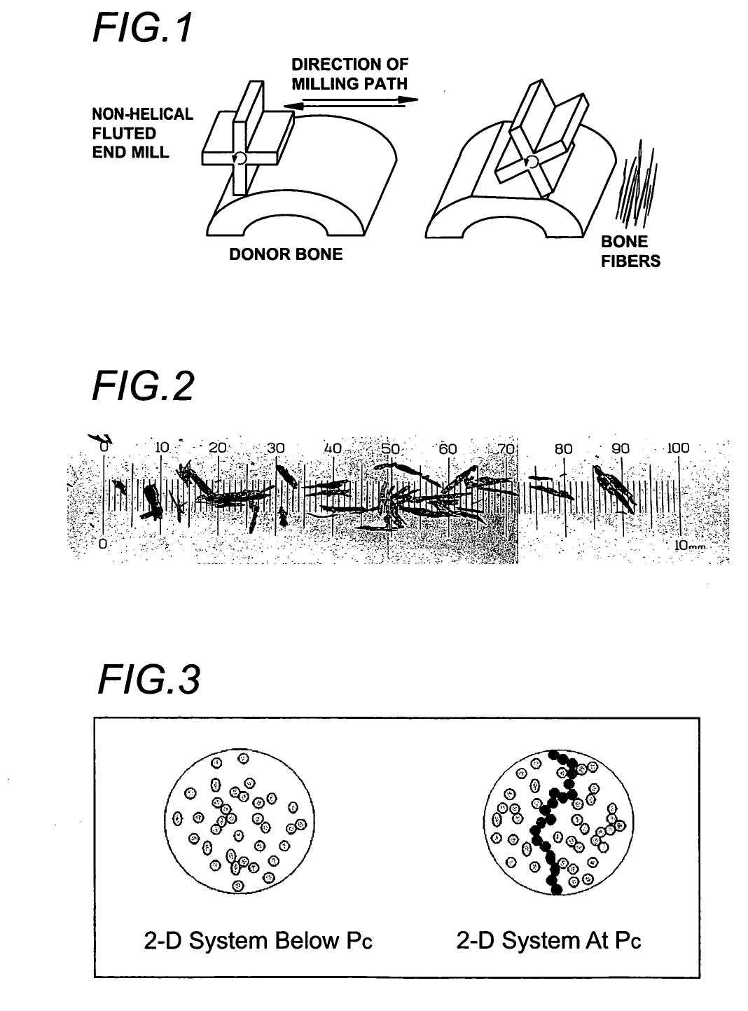

Bone particles may be obtained by milling or shaving sequential surfaces of an entire bone or relatively large section of bone. A non-helical, four fluted end mill may be used to produce fibers having the same orientation as the milled block. Such a mill has straight grooves, or flutes, similar to a reamer, rather than helical flutes resembling a drill bit. During the milling process, the bone may be oriented such that the natural growth pattern (along the long axis) of the piece being milled is along the long axis of the end mill of the milling machine. Multiple passes of the non-helical end mill over the bone results in bone particles having a long axis parallel to that of the original bone (FIGS. 1, 2). Bone particles and fibers with different sizes, dimensions, and aspect ratios may be obtained by adjusting the milling parameters, including sweep speed, bit engagement, rpm, cut depth, etc.

Elongated bone fibers may also be produced using the bone processing mill described in commonly assigned U.S. Pat. No. 5,607,269, the entire contents of which are incorporated herein by reference. Use of this bone mill results in the production of long, thin strips which quickly curl lengthwise to provide helical, tube-like bone particles. Elongated bone particles may be graded into different sizes to reduce or eliminate any less desirable size(s) of particles that may be present. In overall appearance, particles produced using this mill may be described as filaments, fibers, threads, slender or narrow strips, etc. In alternative embodiments, bone fibers and particles may be produced by chipping, rolling, fracturing with liquid nitrogen, chiseling or planeing, broaching, cutting, or splitting along the axis (e.g., as wood is split with a wedge).

The bone fibers may be sieved into different diameter sizes to eliminate any less desirable size(s) of fibers or more evenly dimensioned particles that may be present. In one embodiment, fibers collected from the milling machine may be lyophilized and manually sieved into a range of 500 μm to 300 μm in a particular cross-sectional dimension. One skilled in the art will recognize that the sieving method will determine what aspect must fall within 300-500 μm. Fiber length may be independent of cross-sectional dimension and may be modified by adjusting the bit engagement length, the length of the bit in contact with the bone during the milling operation. Fibers may be an inch long or greater and may be as short as desired, depending on the desired aspect ratio. Fibers less than 50 μm long may increase the likelihood of inflammation depending on the tissues and how the implant degrades. In some instances, particles or fibers of this size may be advantageously included to promote faster bone healing by eliciting a mild inflammatory response. Larger fibers may be further broken into smaller fibers by manually rolling them between the thumb and fingers and then sieved again to select the proper size fibers. Alternatively, fibers may be broken into smaller fibers by pressing or rolling. The resulting fibers may have an aspect ratio of 5:1 to 10:1. Broader or narrower fibers may be obtained by changing sieve grate sizes.

The cell conducting material may be modified in a variety of ways before being incorporated into a composite. For example, fibrous tissues may be frayed to expose protein chains and increase the surface area of the tissue. Rinsing fibrous tissue or partially demineralized bone particles in an alkaline solution, or simply partially or superficially demineralizing bone particles, will fray fibrous proteins within the tissue. For example, bone fibers may be suspended in aqueous solution at a pH of about 10 for about 8 hours, after which the solution is neutralized. One skilled in the art will recognize that this time period may be increased or decreased to adjust the extent of fraying. Agitation, for example, in an ultrasonic bath, may assist in fraying and/or separating collagen fibers, as well as improving penetration of acidic, basic, or other fluids, especially for bony tissues. Alternatively or in addition, bone or inorganic calcium phosphate particles may be mechanically stirred, tumbled, or shaken, with or without the addition of abrasives.

Polymers and fibrous tissues, especially those containing collagen, such as bone and tendon, may be cross-linked before or after incorporation into a composite. A variety of cross-linking techniques suitable for medical applications are well known in the art. For example, compounds like 1-ethyl-3-(3-dimethylaminopropyl)carbodiimide hydrochloride, either alone or in combination with N-hydroxysuccinimide (NHS) will crosslink collagen at physiologic or slightly acidic pH (e.g., in pH 5.4 MES buffer). Acyl azides and genipin, a naturally occurring bicyclic compound including both carboxylate and hydroxyl groups, may also be used to cross-link collagen chains (see Simmons, et al, “Evaluation of collagen cross-linking techniques for the stabilization of tissue matrices,” Biotechnol. Appl. Biochem., 1993, 17:23-29; PCT Publication WO98/19718, the contents of both of which are incorporated herein by reference). Alternatively, hydroxymethyl phosphine groups on collagen may be reacted with the primary and secondary amines on neighboring chains (see U.S. Pat. No. 5,948,386, the entire contents of which are incorporated herein by reference). Standard cross-linking agents such as mono- and dialdehydes, polyepoxy compounds, tanning agents including polyvalent metallic oxides, organic tannins, and other plant derived phenolic oxides, chemicals for esterification or carboxyl groups followed by reaction with hydrazide to form activated acyl azide groups, dicyclohexyl carbodiimide and its derivatives and other heterobifunctional crosslinking agents, hexamethylene diisocyanate, ionizing radiation, and sugars may also be used to cross-link fibrous tissues and polymers. The tissue is then washed to remove all leachable traces of the material. Enzymatic cross-linking agents may also be used. One skilled in the art will easily be able to determine the optimal concentrations of cross-linking agents and incubation times for the desired degree of cross-linking.

The material exploited as a cell conducting phase for use with the invention may also be combined with a desired compound before incorporation into a composite. Exemplary compounds include monomer, prepolymer, telechelic polymers, initiator, and/or biologically active or inactive compounds, including but not limited to biomolecules, bioactive agents, small molecules, inorganic materials and minerals. These compounds may be covalently or non-covalently linked to the cell conducting material, mixed with the cell conducting material prior to or during formation of the composite, or incorporated into the chemical structure of the cell conducting material for release during breakdown.

Exemplary biomolecules that may be combined with the cell conducting material include chemotactic agents and angiogenic factors. Chemotactic factors encourage cell migration into the interior of the composite, increasing the rate of integration of an implant into the surrounding tissue. While this may increase the degradation rate of the composite, it also increases the rate at which the mechanical integrity of the surrounding tissue is restored. The migration of cells into an unvascularized implant is limited by their ability to get nutrients and eliminate waste products. Angiogenic factors may be used to increase the rate of vascularization, providing a blood supply to the interior cells that carries necessary nutrients and removes metabolic byproducts. Alternatively or in addition, it may be desirable to incorporate antibiotics, anti-inflammatory factors, analgesics, bone morphogenic proteins, or growth factors that promote remodeling, collagen production, or bone development into the cell conducting material.

Any of the compounds described above may be attached to ceramic materials through coupling agents. Suitable coupling agents are described in our co-pending application, U.S. Ser. No. 10/681,651, filed Oct. 8, 2003, the contents of which are incorporated herein by reference. Alternatively, the organic phase in bone particles may be exposed using the techniques below and the above compounds linked to the bone particles by reaction with reactive amino acid residues in the exposed collagen or with lipids or carbohydrates also present in bone.

Compounds mixed with or attached to the cell conducting phase may be derivatized to render them inactive for activation at a later time. For example, antibodies may be attached to a growth factor covalently or non-covalently attached to the cell conducting phase. In another example, growth factors or other biomolecules, small molecules, or bioactive agents are encapsulated in a micelle in the composite. The cells degrade the micelle “shell”, releasing the encapsulated material, as they degrade the surrounding composite material. In another embodiment, bone morphogenic proteins are attached to the cell conducting phase in their proform, which must be cleaved before the BMP will exhibit activity. As the cells degrade the cell conducting material, they will also activate the growth factor or other compound.

Bone particles for use with the invention may optionally be superficially, partially, or completely demineralized in order to reduce their inorganic mineral content. In some embodiments, demineralization is used to promote the ability of the matrix to form chemical bonds with the cell conducting phase. Demineralization methods remove the inorganic mineral component of bone, for example, by employing acid solutions. Such methods are well known in the art; see, for example, Reddi, et al., Proc. Nat. Acad. Sci., 1972, 69:1601-1605, the contents of which are incorporated herein by reference. The strength of the acid solution, the shape of the bone particles and the duration of the demineralization treatment will determine the extent of demineralization. Reference in this regard may be made to Lewandrowski, et al., J. Biomed. Mater. Res., 1996, 31: 365-372, the contents of which are also incorporated herein by reference.

In an exemplary demineralization procedure, the bone particles are subjected to an optional defatting/disinfecting step, followed by an acid demineralization step. An exemplary defatting/disinfectant solution is an aqueous solution of ethanol. Ordinarily, at least about 10 to about 40 percent by weight of water (i.e., about 60 to about 90 weight percent of defatting agent such as alcohol) is present in the defatting/disinfecting solution to optimize lipid removal and disinfection and processing time. An exemplary concentration range of the defatting solution is from about 60 to about 85 weight percent alcohol, for example, about 70 weight percent alcohol. Following defatting, the bone particles are immersed in acid over time to effect their demineralization. The acid may also disinfect the bone by killing viruses, vegetative microorganisms, and/or spores. Acids that may be employed in this step include inorganic acids such as hydrochloric acid and organic acids such as peracetic acid. Alternative acids are well known to those skilled in the art. After acid treatment, the demineralized bone particles are rinsed with sterile water to remove residual amounts of acid and raise the pH. The bone particles may be dried, for example, by lyophilization, before being incorporated into the composite. The bone particles may be stored under aseptic conditions until they are used or sterilized using known methods shortly before incorporation into the composite. Additional demineralization methods are well known to those skilled in the art, for example, the method cited in Urist M R, A morphogenetic matrix for differentiation of bone tissue, Calcif Tissue Res. 1970; Suppl:98-101 and Urist M R, Bone: formation by autoinduction, Science. Nov. 12, 1965;150(698):893-9, the contents of both of which are incorporated herein by reference.

In an alternative embodiment, surfaces of bone particles may be lightly demineralized according to the procedures in our commonly owned U.S. patent application Ser. No. 10/285,715, published as U.S. patent Publication No. 20030144743. Even minimal demineralization, for example, of less than 5% removal of the inorganic phase, increases the hydroxylation of bone fibers and the surface concentration of amine groups. Demineralization may be so minimal, for example, less than 1%, that the removal of the calcium phosphate phase is almost undetectable. Rather, the enhanced surface concentration of reactive groups defines the extent of demineralization. This may be measured, for example, by titrating the reactive groups. In one embodiment, in a polymerization reaction that utilizes the exposed allograft surfaces to initiate a reaction, the amount of unreacted monomer remaining may be used to estimate reactivity of the surfaces. Surface reactivity may be assessed by a surrogate mechanical test, such as a peel test of a treated coupon of bone adhering to a polymer. Alternatively or in addition, a portion of the surface of the bone particles may be so demineralized.

Alternatively, the surface of a bone or ceramic particle may be treated to modify its surface composition. For example, nondemineralized bone particles may be rinsed with dilute phosphoric acid (e.g., for 1 to 15 minutes in a 5-50% solution by volume). Phosphoric acid reacts with the mineral component of the bone and coats the particles with dicalcium phosphate dihydrate. Treated surfaces may further be reacted with silane coupling agents as described in our co-pending application Ser. No. 10/681,651, now published as 20050008620, the contents of which are incorporated herein by reference. Alternatively or in addition, bone or ceramic particles may be dried. For example, particles may be lyophilized for varying lengths of time, e.g., about 8 hours, about 12 hours, about 16 hours, about 20 hours, or a day or longer. Moisture may be removed by heating the particles to an elevated temperature, for example, 60° C., 70° C., 80° C., or 90° C., with or without a dessicant. In another embodiment, deorganified bone particles may be used. Deorganified bone particles may be obtained commercially, for example, BIO-OSS™ from Osteohealth, Co. or OSTEOGRAF™ from Dentsply. Alternatively or in addition, bone particles may be partially or completely deorganified using techniques known to those skilled in the art, such as incubation in 5.25% sodium hypochlorite.

Mixtures or combinations of one or more of the above types of bone particles can be employed in the cell conducting phase. For example, one or more of the foregoing types of demineralized bone particles can be employed in combination with nondemineralized bone particles, i.e., bone particles that have not been subjected to a demineralization process. Indeed, mixtures of any of the cell conducting phase components discussed herein are appropriate for use with the invention. The combination of materials may be optimized to provide a particular mechanical property, such as mechanical strength or elastic modulus, or to modify the rate of cellular ingrowth. For example, ceramic or non-demineralized bone particles may increase the strength and stiffness of a composite, while demineralized bone particles may swell more dramatically than mineralized tissue when rehydrated, promoting increased cell migration into the bone particles with respect to non-demineralized bone particles.

Preparation of the Binder Phase

The cell conducting phase is combined with a binder or matrix phase to form a composite. Materials for use in the binder phase may be characterized by one or more of the following: 1) ability to fill spaces around the cell conducting phase; 2) ability to maintain structure and shape under normal physiological loading; 3) biodegradable at a rate consistent with regeneration of the surrounding tissue; 4) ability to be penetrated by cells, either within the cell conducting phase or along the interface between the two phases. Of course, the binder phase may also be a conductor for the cells in question, that is, it may facilitate cell migration along its surface. For example, materials described above as cell conducting materials may be employed in the binder phase.

In one embodiment, the binder phase is a polymer. Polymers for use with the invention may have a variety of textures. Hydrogels provide easy cell infiltration and facilitate diffusion of nutrients until vasculature is developed. They can also conform to the shape of a wound site without the need for machining or molds. Polymers may also be formed as thick mats or felts of non-woven threads. These also promote cell ingrowth but are more mechanically robust than hydrogels. More solid polymers, such as epoxies, thermosets, and thermoplastics, have much greater mechanical strengths and are more easily machined after polymerization.

In one embodiment, biodegradable polymers are used to form composites according to the invention. Exemplary polymers include polylactides, polycaprolactones, polyglycolides, lactide-glycolide copolymers of any ratio (e.g., 85:15, 40:60, 30:70, 25:75, or 20:80), poly(L-lactide-co-D,L-lactide), polyglyconate, polyhydroxybutyrate, polyhydroxyvalerate, polyhydroxybutyrate/valerate copolymers, polyurethanes including glucose-based polyurethanes, and polycarbonates, including tyrosine based polycarbonates, and tyrosine based polyarylates. Additional biodegradable polymers that may be used to form composites according to the invention include poly(arylates), poly(anhydrides), poly(hydroxy acids), polyesters, poly(ortho esters), poly(alkylene oxides), poly(propylene glycol-co fumaric acid), poly(propylene fumerates), polyamides, polyamino acids, polyacetals, poly(dioxanones), poly(vinyl pyrrolidone), biodegradable polycyanoacrylates, biodegradable poly(vinyl alcohols), and polysaccharides. Co-polymers, mixtures, and adducts of any of these polymers may also be employed for use with the invention. In some embodiments, the polymer may be added as a monomer or flowable prepolymer or telechelic polymer and then polymerized once it has infiltrated the cell conducting phase.

Non-biodegradable polymers may also be employed for use with the invention. Exemplary non-biodegradable, yet biocompatible polymers include polystyrene, polyesters, polyureas, poly(vinyl alcohol), polyamides, poly(tetrafluoroethylene), and expanded polytetrafluroethylene (ePTFE), poly(ethylene vinyl acetate), polypropylene, polyacrylate, non-biodegradable polycyanoacrylates, non-biodegradable polyurethanes, mixtures and copolymers of poly(ethyl methacrylate) with tetrahydrofurfuryl methacrylate, polymethacrylate, poly(methyl methacrylate), polyethylene, including ultra high molecular weight polyethylene (UHMWPE), polypyrrole, polyanilines, polythiophene, poly(ethylene oxide), poly(ethylene oxide co-butylene terephthalate), poly ether-ether ketones (PEEK), and polyetherketoneketones (PEKK).

Polymers used in the binder phase or the cell conducting phase may be manipulated to adjust their degradation rates. The degradation rates of polymers are well characterized in the literature (see Handbook of Biodegradable Polymers, Domb, et al., eds., Harwood Academic Publishers, 1997, the entire contents of which are incorporated herein by reference). In addition, increasing the cross-link density of a polymer tends to decrease its degradation rate. The cross-link density of a polymer may be manipulated during polymerization by adding a cross-linking agent or promoter. After polymerization, cross-linking may be increased by exposure to UV light or other radiation. Co-monomers or mixtures of polymers, for example, lactide and glycolide polymers, may be employed to manipulate both degradation rate and mechanical properties.

Exemplary inorganic materials that may be used as a binder phase include degradable ceramics such as calcium phosphate and calcium sulfate. Indeed, any of the ceramic materials described above for use in the cell conducting phase may also be employed in the binder phase. In some embodiments, settable osteogenic materials (e.g. α-BSM, available from ETEX Corp, Cambridge, Mass., Norian SRS, (Skeletal Repair System) available from Norian Corp, Cupertino, Calif., Grafton, available from Osteotech, or Dynaflex, available from Citagenix) is blended with the cell conducting phase. The cement is then allowed to set to produce the composite. The final composite may include a ceramic or a non-ceramic cell conducting phase. Where a non-ceramic phase is used, it may contribute or detract from the mechanical strength of the composite. For example, use of collagen fibers, tendon, or other fibrous materials as the cell conducting phase will increase the strength of the material. Use of a collagen gel will fill pores in a ceramic binder phase without contributing to the mechanical strength of the composite. Where a ceramic cell conducting phase is used, it and/or the binder phase material may be processed so the two materials have different degradation rates. For example, the cell conducting phase may be poorly crystalline or may have growth factors added that promote degradation of the cell conducting phase and production of new tissue. Alternatively or in addition, the binder phase may be highly crystalline or may be heat-treated to increase its crystallinity or grain size.

Alternatively or in addition, synthetic combinations of polymers and inorganic materials may be used in a binder phase. For example, Kryptonite, a polyurethane with a calcium phosphate phase available from Doctors Research Group, Plymouth, Conn., may be used as a binder material. This material may also be used as a cell conducting phase. Of course, natural tissues or extracellular matrix materials such as collagen and tendon may be partially mineralized and employed in composites according to the invention. Exemplary mineralization methods include the use of vacuum or high pressure or chemical deposition of calcium or calcium ceramics.

The materials for use in the binder phase, like those employed in the cell conducting phase, may be modified with a biomolecule, small molecule, bioactive agent, or other compound. Exemplary substances include chemotactic factors, angiogenic factors, analgesics, antibiotics, anti-inflammatory agents, bone morphogenic proteins, and other growth factors that promote cell-directed degradation or remodeling of the binder phase and/or development of new tissue. Such substances may be covalently linked to the binder phase or may be non-covalently associated with the binder phase material. In one embodiment, the added compound is incorporated into the backbone of the polymer and is released as the polymer degrades. Exemplary materials for use in this embodiment include PolymerDrugs, produced by Polymerix, Piscataway, N.J. Alternatively or in addition, the desired substance is simply mixed with the binder phase material before or during preparation of the composite. As for the cell conducting phase, substances incorporated into the binder phase may require activation by local cells.

Combination of the Binder Phase and the Cell Conducting Phase

The cell conducting phase and the binder phase may be combined using standard composite processing techniques or the techniques described in our co-pending U.S. patent application Ser. No. 10/639,912, filed Aug. 12, 2003, and Ser. No.10/735,135, filed Dec. 12, 2003, the contents of both of which are incorporated by reference herein. For example, the binder phase or a binder phase precursor may be combined with the cell conducting phase and injection molded. If a monomeric precursor is used, the binder phase is then polymerized. A partially polymerized precursor may be more completely polymerized or cross-linked after combination with the cell conducting phase. If the binder phase is a material that is flowable under one set of conditions, for example, elevated temperature, and set under a second set of conditions, for example, a lower temperature, then the binder phase in its flowable state is combined with the cell conducting material, injection molded, and allowed to set.

Alternatively or in addition, the binder phase or a binder precursor and the cell conducting phase may be combined and pressed in a Carver press or other compression molding device. Exemplary pressures include pressures ranging from about 1 psi to about 30,000 psi, including around 1,000 psi, around 10,000 psi, around 15,000 psi, around 20,000 psi, or around 25,000 psi. For melt casting applications, heat may be applied in conjunction with the pressure. In some embodiments, any temperature between 20° C. and about 300° C. may be used. One skilled in the art will recognize that higher temperatures may be needed, and that the processing temperature may be optimized to allow the polymer to be processed without damaging other components of the composite. The particular pressure to be used will depend on the materials being pressed. For example, it may be desirable to heat the composite to a temperature in excess of the glass transition temperature of a polymer in the composite. In an alternative embodiment, the composite is formed by injection molding. Where both the binder phase and the cell conducting phase are polymers, if the cell conducting phase has the lower melting point, the composite may be formed by mixing the two components together and molding them at the lower melting point.

In one embodiment, the cell conducting phase and the binder phase are tabletted together before being changed into a mold. For example, the cell conducting phase and binder may be combined and fed into a tabletting apparatus. Any pharmaceutical tablet press may be used, for example, the Minipress available from Globe Pharma, Inc., of New Brunswick, N.J. The tablets enable a more uniform distribution of cell conducting phase in the binder phase. The tabletting process produces tabs of a relatively uniform mass and composition. One or more tablets may be changed into a mold to be pressed into a composite. Tabs of different compositions may be produced to allow production of composites that have regions of different compositions, as described below.

Properties of the composite that influence its final performance include component degradation rate and mechanism, component porosity, and component mechanical properties including strength, fracture toughness, and modulus. While many polymers and ceramics degrade from the surface in, penetration of cells into the interior of the composite can increase the overall degradation rate and cause more uniform degradation across a cross-section of the composite material. Both the inherent porosity of the composite and induced pathways influence the overall composite degradation rate by facilitating the infiltration of cells into the composite. As is well known, the mechanical properties of a composite are influenced by the mechanical properties of the phases as well as the interaction between the phases. For example, hard inclusions in a polymeric phase can add strength to the final composite, while fibrous inclusions well-bonded to a ceramic phase add fracture toughness. The description below is meant to offer a guide to the skilled artisan as to how to manipulate the transformation and/or degradation rate of a composite produced according to the invention and how to adjust the mechanical, chemical, biological, and other properties of a composite having a particular composition.

One advantage of these composites is that, following implantation into a living host, they either completely or partially transform into host tissue. Host cells are able to penetrate and stabilize the composite with host tissue prior to substantial resorption or degradation of the overall construct or its components. Transformation may occur through the active replacement of all or portions of the composite construct by the penetrating cells, or by cellular penetration into the construct (e.g., along component interfaces) with subsequent replacement or degradation of the composite or one or more of its components. As described herein, cells and tissue are considered to have reached the interior of an implant when they have penetrated a sufficient distance into the implant that nutrients cannot diffuse to the cells from the surrounding tissue. That is, vascularity within the implant is needed to provide nutrition to and remove wastes from cells. For soft tissues, this limit may be 0.5 mm, 1 mm, or more depending on the tissue, but it may be much less for mineralized tissue. Still, in some embodiments, it may be desirable that endogenous cells and/or tissue penetrate to a distance of at least 1 mm, 2 mm, 3 mm, 4 mm, 5 mm >7.5 mm, or more from a surface of the implant. In alternative embodiments, the desired degree of penetration may be defined by a percent of a particular radius of an implant. For example, cells and/or tissue may penetrate to a distance of at least 5%, 10%, 20%, 30%, 40%, 50%, 60%, 70%, 80%, 90%, or 99% of a radius of an implant. For non-spheric implants, it is not necessary that the radius in question be the longest or shortest radius of the implant—the desired radius may depend on the implant site.

The inventive composites are partially characterized by their ability to transform into host tissue in the absence of any significant inherent macroporosity. This property distinguishes the invention from traditional tissue engineering matrices which either require the incorporation of macropores to facilitate tissue ingrowth, or alternatively allow tissue ingrowth only as the polymer itself resorbs and provides space for tissue growth to occur.

In some embodiments, the composite is intended to completely transform into host tissue with no or minimal remnants of the initial composite remaining. However, other applications may require an initial phase of long term partial transformation (e.g., between 5 and 50% by volume over 1 year) prior to the completion of the transformation process. In some cases, partial transformation is desired as the final endpoint (e.g., anchoring constructs prepared with tissue and non-resorbable polymers). In these cases, a nonresorbable or partially resorbable matrix or cell conducting surface may be employed.

The degree of transformation of a composite may be expressed as the amount of new tissue present in the tissue/composite construct as a function of the amount of composite remaining. Thus, transformation can be expressed on either a volume, weight, weight to volume, or volume to weight basis. Standard histological or imaging methods may be used to determine tissue amounts. In some cases, biochemical surrogates for tissue may be used (e.g., levels of alkaline phosphatase, collagen, gene expression, etc). Similar approaches may also be used for establishing the amount of residual composite at a given time point.

The transformation process may be initiated by penetration of cellular and/or tissue elements into the composite. Exemplary tissue elements include vascular elements that ensure respiratory and nutritive needs for the transformation of the composite into tissue are met. During this process, the required physical and mechanical properties of the composite construct are maintained. In some embodiments, only after tissue ingress into the construct has progressed to an extent that ensures the required properties are retained by the construct does significant degradation of the overall composite begin. The degradation rate of the binder phase and the distribution of the cell conducting phase may be adjusted to control the rate of change of the mechanical properties of the composite.

The cell conducting phase of the composite may provide a continuous path for cell migration and tissue ingrowth across the entire composite or a portion of the composite. In many embodiments, the cell conducting phase is comprised of particulate material. Use of particulates facilitates molding structures fabricated with the composite. Furthermore, contiguity of the components of the cell conducting phase promotes transformation of the composite. In order to optimize the contiguity of the cell conducting phase, we exploited percolation theory. Percolation theory addresses the mathematical modeling of connectivity (contiguous adjacency) of randomly distributed components in a system. The percolation threshold (P_{c}) is the volume fraction at which adjacent randomly distributed components will form one path spanning the system (FIG. 3). P_{c} is a function of included component shape and puts a lower limit on the volume fraction of inclusions of a specific shape needed to achieve system-spanning interconnectivity. Below P_{c}, there is not enough of the included component to provide a continuous path across the system. Further details of percolation theory useful for the production of the inventive composites may be found in Garboczi, et al., Geometrical Percolation Threshold of Overlapping Ellipsoids, Phys. Rev. E, 1995, 52(1):819, incorporated herein by reference.

Percolation theory provides a framework for designing a composite, but strict adherence to the theory is not necessarily the most efficient method of designing certain implants. For example, once the system reaches P_{c}, there will still be isolated clusters of the included component. Increasing the proportion of the included component reduces the percentage of the included component that is in these isolated clusters. In addition, percolation theory is based on a random distribution of particles. A continuous path from one side of an implant to another may not necessarily traverse the interior of the system. Furthermore, models based on percolation theory often employ solid ellipsoidal particles. Still, it is possible to use the aspect ratio of a non-ellipsoidal bone particle to determine a lower limit for the amount of a randomly distributed cell conducting phase needed to provide a continuous path spanning an implant. The use of porous particles to form a composite is discussed in Example 4.

To increase the probability that the cell conducting phase provides a path to the interior of the composite, it may be necessary to increase the proportion of the cell conducting phase beyond the percolation threshold. For this reason, in some embodiments, it may be more useful to discuss the percentage of cell conducting phase that is in connected networks rather than the percentage of cell conducting phase required to span the system.

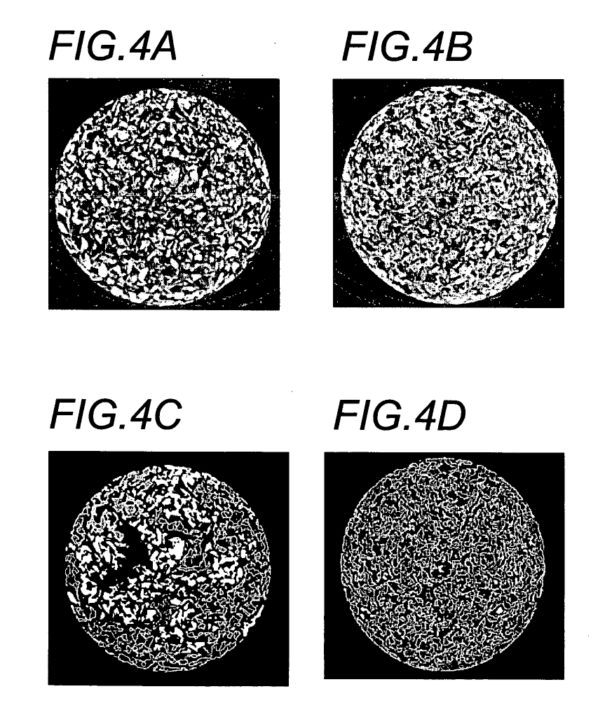

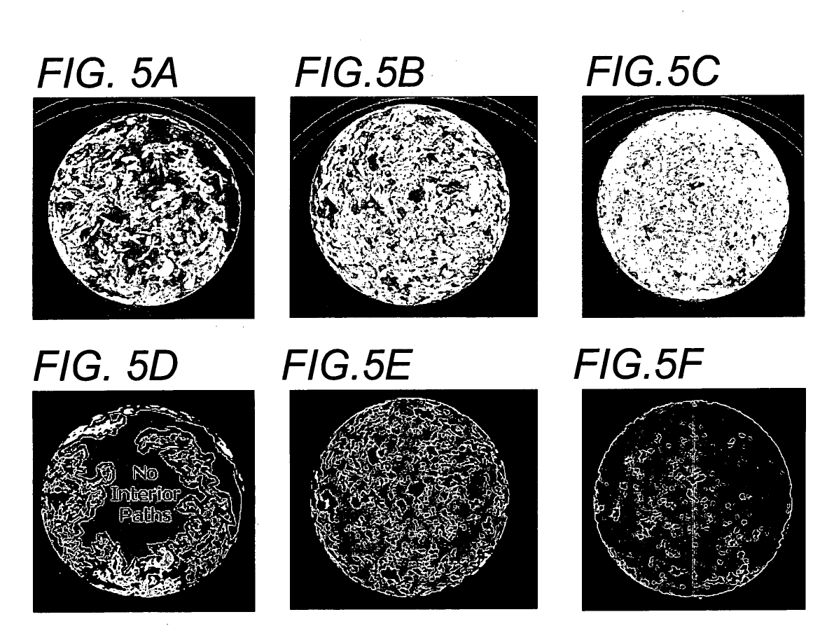

As a result, some experimentation may be needed to design an implant having a desired level of connectivity. Given a particular particle size distribution in the cell conducting phase, composites may be fabricated with differing proportions of the cell conducting phase and binder phase. Cross-sections of these composites may then be examined using micro-computed tomography (micro CT) techniques. These cross-sections may then be examined to determine the connectivity of the cell conducting phase from the surface to the interior of the composite. FIGS. 4 and 5 contrast micro CT images for cross-sections of composites employing particles with an aspect ratio of 2:1 and fibers with an aspect ratio of about 8:1. The figures show that, in two dimensions, small increases of the proportion of the cell conducting phase dramatically increase the continuity of the phase for both particles and fibers. One method of quantifying the continuity of the phase is by calculating how much of a cross-sectional area of an implant the largest connected cluster of the cell conducting phase occupies. A portion of the cell conducting phase is a connected cluster if it provides a continuous path from any point in the cluster to any other point in the cluster. For example, a group of bone particles within a composite in mechanical communication with one another is a connected cluster. The greater the area fraction occupied by a cluster, the more likely it is that the cluster provides a path not only to the interior of the implant but to the center of the implant. Table 1 shows the largest connected cluster area fractions for the cross-sections shown in FIGS. 4 and 5. If the particle distribution in the implant is isotropic, then the area fraction also gives an idea of the volume fraction of an implant that cells and tissue can reach, so long as cells have access to at least one particle in a connected cluster. The micro CT images may be used to identify the composition with the desired connectivity for the particular application.

TABLE 1

| | |
|---|---|
| | Area percent |
| | occupied |
| Bone/polymer | by largest |
| ratio by | connected cluster |

| weight | Partcles | Fibers |
|---|---|---|
| 50/50 | | 11.4 |
| 60/40 | 12.9 | 72.2 |
| 70/30 | 59.7 | 95.7 |

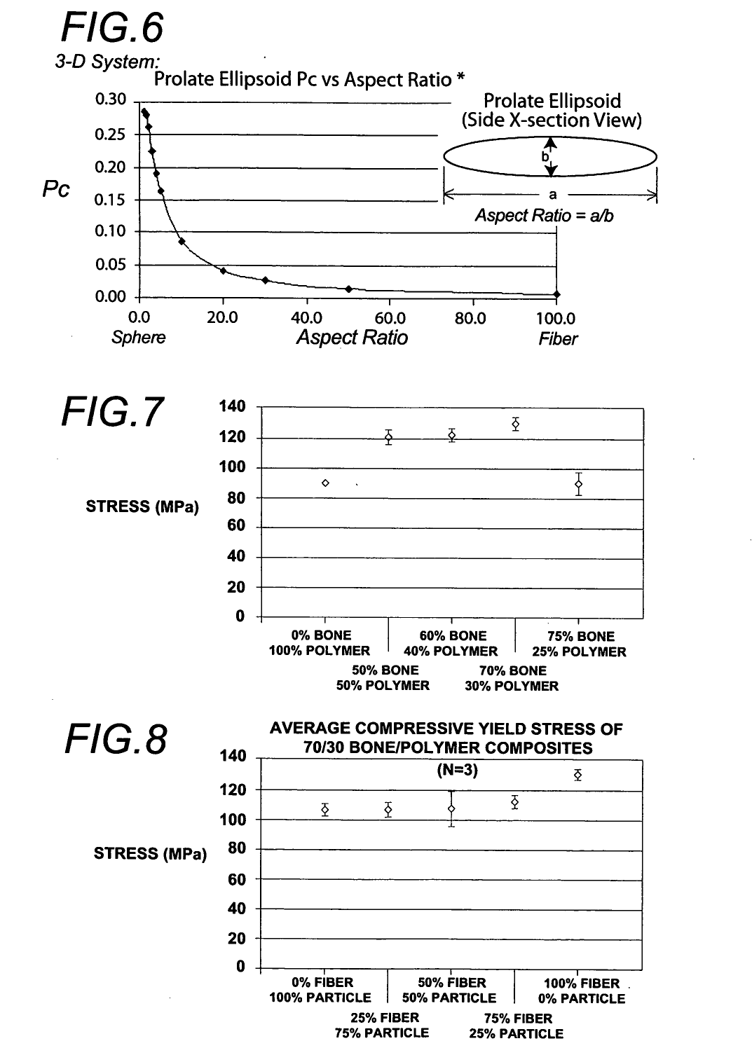

In one embodiment, the cell conducting phase provides as many paths to the interior of the composite implant (bone-to-bone filler connections) as possible, without compromising the implant's mechanical properties. For this embodiment, cell conducting phase components with shapes that give high connectivity at low volume fractions are desirable. Fibers fulfill this criterion (FIG. 6). In one embodiment, bone fibers used to produce composites according to the invention are sufficiently long that about 15% to about 20% or about 30% by weight is sufficient to provide a path across an implant or to the interior of an implant from the surface. Depending on the aspect ratio and size distribution, as much as 50%, 60%, or 70% of bone particles or fibers may be required to provide a desired path. Indeed, depending on the size of the final implant and the aspect ratio of the fibers, a single bone fiber may be sufficient to provide a path not only to the center of an implant but all the way across a diameter of the implant.

Increasing the volume fraction of the cell conducting phase within the matrix increases the continuity of the cell conducting phase and allows cells to reach more of the interior of the composite without blockage by the binder phase. Too great a volume fraction of the cell conducting phase may degrade the mechanical properties if there is not sufficient binder phase to hold the particles and pieces of the cell conducting material together. The fraction of a ceramic cell conducting phase (including bone particles) may be between about 60% and about 85%, for example, about 70 percent, about 75 percent, or about 80% by weight. The desired volume fraction of the cell conducting phase may depend on the shape of the cell conducting material because of the effect of particle shape on both connectivity and mechanical properties. In one embodiment, the volume fraction of the cell conducting phase is at least about 27%, about 35%, about 40%, or about 50%. To optimize biological performance of the implant, the proportion of the cell-conducting phase may be as high as practicable. In practice, this ideal may be limited by other considerations. For instance, a minimum amount of the binding phase may be necessary to provide particular mechanical properties to the implant. Likewise, certain forming processes can be limited in their ability to form an implant containing high levels of solids. Evenly proportioned particles may have less surface area for a given volume than elongated particles. A single elongated particle or a piece of tendon can reach deep into the composite material, while an evenly proportioned particle of the same volume does not provide a path for deep cell infiltration.

Specific paths across and into the composite may be created without necessarily relying on the random arrangement of the cell conducting material. For example, a gradient of the cell conducting material may be established so that there is a larger proportion of the cell conducting material at the periphery of the composite and a smaller proportion in the interior. As discussed above, such a gradient may be established by using tablets of different compositions in different regions of a mold. In one embodiment, a high concentration of cell conducting material at the surface of a composite may encourage rapid ongrowth, while only tendrils of the cell conducting material extend into the composite. These tendrils enable native tissue to extend across the implant without occupying a significant volume of the implant.

In an alternative embodiment, the composite exhibits regional continuity. For example, fingers of a cell conducting material penetrate into the binder phase from a surface of the composite. These fingers may take on any shape and may themselves exhibit a composition gradient with respect to the cell conducting material or some other additive as they proceed into the composite from the surface. These fingers would provide a mechanism to anchor a non-resorbable implant to bone tissue. This technique may be used for soft tissue anchors, fixing joint implants, providing anchorage of bone cements in revision surgeries for joint implants, and anchoring plastic prosthetics, such as finger tips, chins, etc. In one embodiment, a polymeric interbody device (e.g., an artificial disk) is anchored to a vertebral body using a gradient of cell conducting material in the portion of the implant adjacent to the vertebral body endplate. A high concentration of cell conducting material at the surface of the implant may encourage migration of osteoblasts and other cells onto the fingers, followed by rapid bony ongrowth to, for example, the surface of particles of the cell conducting material.

The rate of cell infiltration may be increased by adding an active agent to either the binder phase or the cell conducting phase. The addition of biomolecules, small molecules, and bioactive agents to the cell conducting phase and the binder phase is discussed above. The addition of appropriate regulators, for example, bone morphogenic proteins, to the binder phase, will upregulate cell-based degradation of the binder phase and formation of new tissue such as bone. Gradients of chemotactic factors or other substances in the cell conducting phase, the binder phase, or both, can also encourage cellular infiltration. Such gradients may be created by adding the chemotactic factor at varying rates as the flowable binder precursor and cell conducting phase are being introduced into a mold. Indeed, different biomolecules, small molecules, or bioactive agents may be introduced in different sections of the composite in the same manner by adding the appropriate substance to a feed being introduced into a mold.

Where a mold is manually charged, gradients or variations in composition may be introduced by preparing different compositions of the cell conducting material, the binder precursor, or both, with different concentrations of a particular substance or different substances. The desired substance is added as a particular section of the mold is filled. For example, the bottom of a mold may be charged with a cell conducting material having an anti-inflammatory agent, while central portions of the mold are charged with a mixture of the binder precursor and a cell conducting material having higher concentrations of a chemotactic factor. Alternatively or in addition, a temporary insert may be used to vertically separate sections of a mold, with different precursor compositions in each section.

The same techniques may be used to vary the ratio of the cell conducting phase across an implant. Alternatively or in addition, the materials themselves may be manipulated to achieve this. For example,.in a composite comprising bone fibers and a high melt viscosity polymer, the bone fibers may tend to travel towards the surface of the implant during formation.

The desired variation, if any, in composition depends in part on the implant type. If the implant is to go into a large segmental defect, rapidly transforming ends allow the formation of a fracture callus and facilitate early attachment, while a slower-transforming or angiogenic central region helps maintain the mechanical strength of the implant as transformation proceeds inward from the healed edges. Likewise, for a spinal implant, a strong axial pillar of allograft bone may be embedded within a weaker, yet biologically active matrix of binder plus a cell conducting material. Alternatively or in addition, cells may be excluded from a portion of the implant by filling a region of the implant with only a non-cell conductive binder phase, or by putting a “firebreak” of a non-cell conductive binder material around a protected region.

The degradation rates of polymers in composites produced according to the invention also depends on the permeability of the polymer to cells. Porous mats and hydrogels are relatively permeable to cells and will also degrade faster than more solid polymers. Such materials may be used in composites in which it is desirable to have more rapid cell ingrowth but slow overall degradation rates. Cells will migrate along the cell conducting phase quickly, but degradation of the binder phase will proceed at a slower pace. As a result, the composite is integrated quickly, before any decreases in mechanical strength due to degradation of the binder phase. However, large amounts of porosity, particularly, macropores, limit the ultimate mechanical strength attainable by the composites. The degradation rate may also be influenced by the continuity and the distribution of the cell conducting phase. In a composite where cells penetrate along the surface of or through the cell conducting phase, phase continuity helps cells infiltrate all portions of the cell conducting phase. Where the cell conducting phase is less continuous, the surrounding binder phase may need to biodegrade or resorb in order for cells to gain access to other sections of the cell conducting phase.

The degradation rates of ceramic components, including bone, of composites according to the invention depend on both the composition and the crystallinity. Carbonate-substituted hydroxyapatite promotes new bone development more quickly than unsubstituted hydroxyapatite. Amorphous calcium ceramic phases degrade more quickly than crystalline ceramic phases. Heat treatment increases the crystallinity of calcium phosphates and other ceramics and decreases their dissolution rate.

Extracellular matrix components or tissue-derived materials employed in the practice of the invention may also be treated to influence their remodeling rate. For example, collagen or collagen-containing tissues may be cross-linked using the techniques described above to reduce their degradation rate. Alternatively or in addition, they may be partially mineralized or derivatized with other biomolecules or bioactive agents.

The mechanical properties of the composite are influenced not only by the composition of the binder phase and the cell conducting phase but their geometric arrangement with respect to one another. Both particulate cell conductors and fibrous cell conductors have the potential to increase the stiffness of a composite. Elongated particles or materials may be aligned in the composite or may be randomly oriented. The alignment of an elongated cell conducting phase may be uniform throughout the composite or may be varied. For example, elongated particles may be lined up parallel to one another or in a two or three dimensional cross-hatch pattern. Layers of aligned cell-conducting phase materials may be arranged such that the layers alternate or rotate in orientation. The layers may form a continuous rotation through the third dimension (e.g., a rotated plywood structure, described in Neville A C, Biology of Fibrous Composites, Cambridge University Press, 1993, incorporated herein by reference). Likewise, the layers may alternative in orientation, with each layer positioned to best resist the most likely loading modes on the implant. Examples include 45°/−45°/90° alternations. Such alternating laminate composites are described in Gibson R F, Principles of Composite Material Mechanics, McGraw-Hill Series in Aeronautical and Aerospace Engineering, ed. Anderson Jr. J D., McGraw-Hill, 1994, incorporated herein by reference. Additionally, discontinuous oriented or random fiber composites can be configured, as described in Gibson (1994). Alternatively, elongated particles may be oriented in a spiral or other shape. The arrangement or volume fraction of the cell-conducting phase, or even the size and shape of its components, may be varied along a dimension of the composite material. For example, a composite may have a gradient of fibrous particles around its exterior and more evenly proportioned particles in the center. In one embodiment, the exterior portions of the composite have a higher concentration of chemotactic factors and other factors that may promote degradation of the cell conducting phase or the binder phase, while the interior portions of the composite have a higher concentration of factors that promote bone formation. The cell-conducting phase may be oriented to direct tissue growth, for instance, across a gap or between adjacent spinal processes in a posterolateral fusion.

Processing of Composite

The surface of the composite may be modified after the binder and cell conducting phases are combined. The binder phase tends to flow around the cell conducting phase during processing, so that the surface of the composite is mostly binder material. Abrasion methods are useful for exposing the cell conducting phase and provide surface roughness. Machining or cutting the composite will also expose the cell conducting phase. Both the mechanical texture of the composite and the exposure of the osteoconductive phase to surrounding tissue may facilitate initial cell migration onto and into the composite material. Surface roughening may be accomplished mechanically, for example, through sanding, tumbling with a hard material such as sand, or the use of a pulsatile wave (e.g., the composite is conveyed above a liquid bath, and waves pulse the liquid into crests that contact the material). The desired surface texture may also be achieved using other machining methods, including but not limited to grinding, milling, cutting, broaching, drilling, laser etching, water cutting, and sand blasting. Chemical treatments may be used as well. The use of solvents capable of solubilizing the binder phase material can be used to improve exposure of the cell conducting phase. Organic solvents such as acetone will remove some polymers from the surface of a material, while dilute acids may be used to roughen inorganic materials. Implants containing hydrolytically degradable polymers may be treated with water to pre-degrade the surface before implantation. The surface of the composite may also be modified to postpone cellular ingrowth. For example, the composite may be coated with a rapidly degradable or soluble material, or regions may be masked so that the cell-conductive phase is not exposed in certain regions during abrasive grinding, tumbling, sanding, etc. operations. Cells can migrate along the cell conducting phase after the coating is degraded or dissolved. The rate at which the surface of the composite is exposed may be adjusted such that the cell conducting phase is revealed at a particular pointing in the healing cascade.

Of course, the composite may also be machined. In one embodiment, the composite is machined into a block which can be completely infiltrated by tissue within a predetermined time period. Alternatively, the composite may be machined into any desired shape and size. Exemplary shapes include sheet, plate, particle, sphere, hemisphere strand, coiled strand, capillary network, film, fiber, mesh, disk, cone, portion of a cone, pin, screw, tube, cup, tooth, tooth root, bone, portion of bone, strut, wedge, portion of wedge, cylinder, threaded cylinder, rod, hinge, rivet, anchor, spheroid, ellipsoid, oblate spheroid, prolate ellipsoid, hyperbolic paraboloid. Composites may also be formed into the shape of a bone or a portion of a bone. Exemplary bones whose shape the composite may match in whole or in part (and which may be repaired or replaced using the techniques of the invention) include ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, incus, malleus, stapes, ilium, ischium, pubis, femur, tibia, fibula, patella, calcaneus, tarsal and metatarsal bones. In another embodiment, the composite is formed as a plate or similar support, including but not limited to an I-shape to be placed between teeth for intra-bony defects, a crescent apron for single site use, a rectangular bib for defects including both the buccal and lingual alveolar ridges, neutralization plates, spoon plates, condylar plates, clover leaf plates, compression plates, bridge plates, wave plates, etc. Partial tubular as well as flat plates may be fabricated using the techniques provided by the invention. Composites may be molded into any of these shapes as well, obviating a machining step or reducing the amount of machining needed. The machining process often exposes the cell-conducting phase.

In an alternative embodiment, bores or holes may be introduced into the composite. Such holes may be drilled after the composite is formed. Alternatively or in addition, the mold may be formed with pegs to introduce holes into the composite. Such holes may be used to provide a n anchor for sutures, screws, or other fasteners. Of course, cells will also migrate into the hoe after implantation. The drilling or boring process often exposes the cell-conducting phase.

Whether or not the composite is expected to be completely infiltrated within a predetermined period of time, it may have sufficient mechanical strength to withstand physiological loads until it is fully transformed. In one embodiment, the composite has a yield strength in aqueous environments of about 40 MPa or greater and an initial wet stiffness of 1 GPa or greater. Alternatively or in addition, the composite may experience wet creep of less than about 10.5% or less than 2 mm height loss in the intervertebral body space. Fatigue life may be greater than 1.25 million cycles, for example, at least 3 million cycles, at 25 MPa. As the material degrades, it may retain some mechanical strength, for example, having at least 25 MPa residual strength after 6 months in vivo. Alternatively, they may maintain at least 70% of their original strength after 24 weeks. The degradation rate of the binder phase may be matched to the rate at which surrounding tissue can interpenetrate the implant or remodel injured tissue surrounding the implant. For example, at least 25% of the implant is transformed or penetrated by cells in 4, 8, 16, or 24 weeks, at least 50% of the implant is transformed or penetrated by cells in 4, 8, 16, or 24 weeks, at least 75% of the implant is transformed or penetrated by cells in 4, 8, 16, or 24 weeks, or at least 90% of the implant is transformed or penetrated by cells in 4,8, 16, or 24 weeks. The desired mechanical properties depend on the specific implant application. For example, a bone void filler can transform quickly and need not have high mechanical strength, while a lumbar interbody implant may need to exhibit substantially higher compressive and fatigue strength as it is transformed.

The mechanical properties desired for the composite and implants fabricated from the composite depends on the application in which the implant will be used. For example, implants that will be subjected to torsional stresses, such as spruce, may have stiffness of 0.5 GPa or greater and torsional strength of at least 25 MPa, for example, at least 30, 35, 40, 45, or 50 MPa. Plates that will be subject to bending stresses may have stiffnesses of at least 2 GPa, for example at least 5, 10, or 20 GPa. Plates may be able to withstand bending stresses of at least 50 MPa (outer fiber stress in center of 3 point bending sample), for example, at least 100, 250, or 500 MPa. Rods may be employed in a variety of implants and tissue locations. In some embodiments, rods have a tensile strength of at least 100 MPa, for example, at least 150 or 200 MPa, bending strengths of at least 100 MPa, for example, at least 150 or 200 MPa, and torsional strengths of at least 50 MPa, for example, at least 75 or 100 MPa. In some embodiments, rods may have stiffnesses of at least 5 GPa, for example at least 7 or 10 GPa in tension, torsion, or flexion. For all these implants, the implant may retain between 70 and 100% of its strength at eight weeks post-implantation and have at least 95% resorption after two years. For example, the implant site may maintain 90 to 100% strength to eight weeks post-implantation, with full mass loss of the implant at about one year. Specific implant sites may have their own requirements. For example a lumbar interbody device may have a compressive strength of at least 40 MPa and initial stiffness of 1 GPa and be able to withstand 1.25 million cycles at 25 MPa. Because the lumbar device is subjected to almost constant stress, it may also have a target creep of less than 10.5% maximum asymptotic strain after one week of being loaded at 25 MPa at 37 degrees C. The device may still have a strength of at least 25 MPa after six months in situ.

In some embodiments, especially where the expected loads on the implant are expected to be less (e.g, cranial implants), the transformation rate of the implant may be increased by adding porosity to the implant. For example, the composite may be formed with a porogen such as a salt or foaming agent. Alternatively, a gas may be introduced to the composite as it is formed, introducing air bubbles to the final product. Such agents may be biocompatible to reduce the risk from incomplete removal of the porogen from the composite. The porosity will increase the exposed surface area of the cell conducting phase within the implant and may increase the number of pathways for cells to penetrate the implant.

Post-Implantation

Once the composite is implanted, cells migrate into it from the surrounding tissue. Cells that migrate to the cell conducting phase will infiltrate and degrade the material more quickly than cells that migrate to a non-conducting phase surface. In one embodiment, cells infiltrate the composite by actively degrading the cell conducting phase, the binder phase, or both. Cells may degrade the entire cross section of the cell conducting material or may simply create a path along the interface of the cell conducting phase and the binder phase. In one embodiment, the composite is essentially solid, but a tissue-based cell conducting phase has inherent porosity of a size appropriate for cell migration. For example, the tissue may have pores or spaces ranging in size from 100 to 500 μm or greater. Alternatively or in addition, the tissue may have pores or spaces less than 100 μm across, for example, less than 25, 50, 25, or 10 μm. In many embodiments employing a solvent or thermal cast polymeric matrix, many or all of these pores are filled with binder phase during the casting process. As discussed in Example 4, this may enhance to the mechanical properties of the implant. In an alternative embodiment, cells do not simply migrate into the composite but actively create a path by degrading it. In another embodiment, the conducting phase displays a dimensional change after implantation. For example, the cell conducting phase or binder phase may swell upon exposure to an aqueous environment. Alternatively or in addition, the composition of the cell conducting phase may be such that the cells continue to travel into the composite rather than settling near the surface and creating tissue. For example, the cell conducting material may be highly osteoconductive without being inductive, or chemotactic factors and factors that discourage tissue synthesis may be attached to the cell conducting phase. As the cells “dig” their way into the composite, the composition of the material may change to begin upregulating tissue formation. As a result, cells that initially reach the composite travel well into the composite before producing tissue, allowing later-arriving cells to migrate up the paths formed by their predecessors into the composite. In some embodiments, the composite may help regulate the local chemical composition during degradation. For example, polymer degradation may produce acidic breakdown products. The decrease in pH will demineralize any bone particles in the vicinity and/or dissolve ceramics, neutralizing the acid and increasing the osteoinductivity of the bone.

The use of composites that provide paths for cellular ingrowth using osteoconductive materials rather than inherent porosity allows implants fabricated from these composites to exhibit higher yield strengths post-implantation and to be used in load bearing applications. Furthermore, patients with these implants can return to weight-bearing activities sooner than they otherwise might be able to. In some embodiments, implants may have yield strengths in compression approaching those of cortical bone, for example, greater than 80 MPa, greater than 130 MPa, and as high as 200 MPa. In bone, yield strength depends on the direction of loading, and implants may be fabricated to mimic this anisotropy or to have more isotropic mechanical properties. Of course, the desired strength of the implant also depends on the implant site. Implants in the leg will experience greater loads and different loading modes than an implant in the skull.

### EXAMPLES

### Example 1

Compression and fatigue data was generated from samples containing fully mineralized bone fibers and particles. Bone fibers were generated by milling cortical bone with a non-helical four fluted mill and were 300 μm-500 μm wide, with aspect ratios between 3:1 and 10:1. The bone mill was operated at 760 rpm and a pass speed of 0.5 ips. The depth on each pass was 0.02 in. Evenly dimensioned bone particles were generated by grinding and sieving and were 200-500 μm in diameter, with aspect ratios between 1:1 and 2:1. All reported ratios are by weight. The method used to form test samples, apart from bone shape (fiber or particles), was the same. The bone pieces were dried and combined with sufficient polyDTE-carbonate in the assigned proportion (see FIGS. 7 and 8) to make 1-2.5 gram of pre-composite material. The bone/polyDTE-carbonate mixture was charged into a mold and pressurized. The temperature was raised to 110° C. at a rate of 3-4° C./min., following which the mold was repressurized to 2000 lbs. The mold was then cooled to 60° C. and the composite removed.

Test samples were formed from dried polymer and bone components. Test samples were cylindrical, with a diameter of 11.2 mm and a length of ˜11 mm. Test samples were rehydrated in phosphate buffered saline solution (PBS) at 37° C. for 24 hours prior to mechanical testing. Compression tests were displacement controlled at a rate of 25 mm/minute. Fatigue tests were load controlled, including a 25 MPa maximum stress and a minimum stress of 2.5 MPa at a frequency of 5 Hz. Fatigue test samples were immersed in an antibiotic saline solution at 37° C. during testing. Fatigue failure was defined as the point where 10% maximum strain occurred. Fatigue tests were stopped if failure had not occurred after ˜2 million cycles.

The results of the compression tests demonstrate that bone fibers can be used to produce enhanced polymer/bone composite samples with wet compressive strengths higher than that of the polymer alone. This holds true up to a bone fiber content of around 75% by weight (FIG. 7). Fibers can enhance the composite strength of composites that also contain evenly dimensioned bone particles. In general, for polymer/bone samples that all contain 70% total bone by weight, compressive strength is enhanced for bone fractions containing >50% fibers with respect to composites containing only particles (FIG. 8). Within these ranges, variation of fiber and particle blends along with polymer/bone ratios can be used to control compressive mechanical properties. At comparable polymer/bone ratios, fiber-containing samples exhibited enhanced compressive fatigue properties. In fatigue tests, fiber reinforced samples showed a higher number of cycles to failure (>2 million cycles) when compared to composites containing more evenly dimensioned bone particles (˜400,000 to 800,000 cycles) (Table 2).

TABLE 2
Fatigue cycles to failure of bone/DTE composites

| Bone/Polymer | Type of | Cycles |
|---|---|---|
| Weight Ratio | Bone Used | To Failure |
| 75%:25% | fiber | 2053750* |
| 75%:25% | fiber | 2006000+ |
| 70%:30% | fiber | 2030750* |
| 70%:30% | fiber | 2048402* |
| 50%:50% | fiber | 1944750* |
| 75%:25% | particle | 440250+ |
| 65%:35% | particle | 87750+ |

*= test stopped
+= sample failed

### Example 2

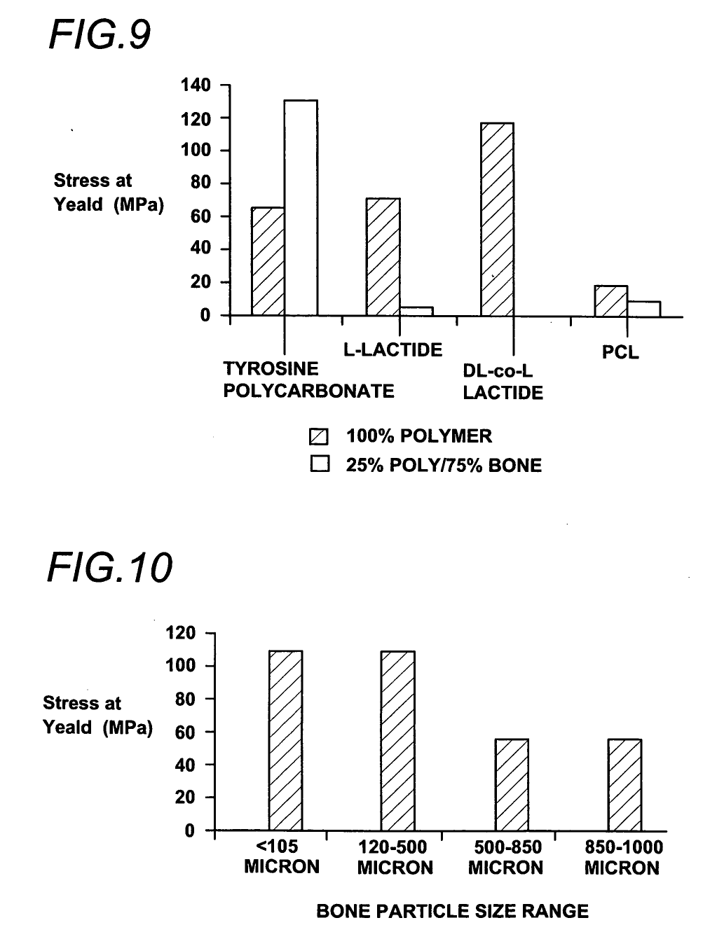

Composite samples were produced from cortical bone particles having relatively even dimensions. Particles from diaphysical bone were ground, lyophilized, and sieved to desired size ranges as discussed below. The particles were combined with one of polyDTE-carbonate, poly L-lactide, poly(DL-co-L-lactide), and polycaprolactone (PCL) in a ratio of 75:25 by weight. The mixtures were molded in a cylindrical press at 13,600 psi and a temperature 15° C. greater than the glass transition temperature of the polymer. Polymeric materials were evaluated for wet compressive strength, both alone and in combination with allograft bone particles (FIG. 9). The lactide materials displayed good wet strength, but when combined with bone (75% bone/25% polymer, by weight), their strength deteriorated substantially. By contrast, polyDTE-carbonate exhibited significant reinforcement from the bone particles (FIG. 9). The composite withstood 3 million cycles with accelerated cyclic loading at 25 MPa (MTS 858 MiniBionix test system, MTS, Eden Prairie, Minn.).

The size of bone particles used in the composite significantly affected material properties (FIG. 10), with smaller particle sizes more effectively reinforcing the composite. Where the release of small embedded bone particles may lead to an inflammatory response, the particle size may be optimized to balance mechanical properties with the desired physiological response.

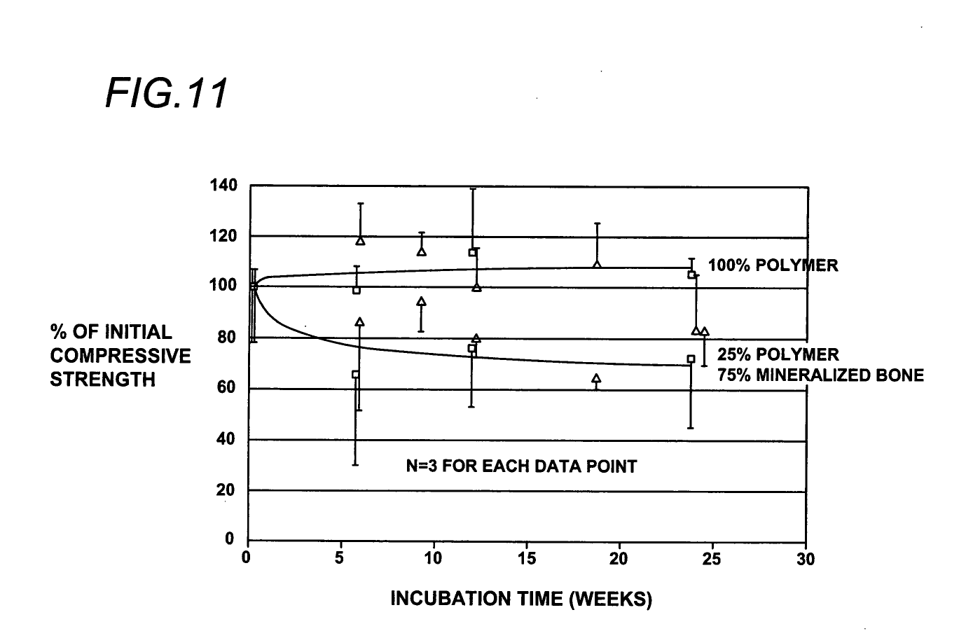

When samples of polyDTE-carbonate/mineralized bone composites were implanted into rabbit paravertebral sites for periods up to 24 weeks, they maintained approximately 70% of the original implant strength (FIG. 11). They also demonstrated a similar loss to paired samples that were tested after soaking in a saline solution in vitro for the same periods. This finding suggests that hydrolysis is the primary mode of degradation (Tangpasuthadol, et al., Hydrolytic degradation of tyrosine derived polycarbonates, a class of new biomaterials. Part II: 3-yr study of polymeric devices. Biomaterials, 2000, 21: 2379 87; Tangpasuthadol, et al., Hydrolytic degradation of tyrosine derived polycarbonates, a class of new biomaterials. Part I: study of model compounds. Biomaterials, 2000, 21: 2371 8), and that enzyme or cellular mediated processes contribute little to polymer breakdown in this period.

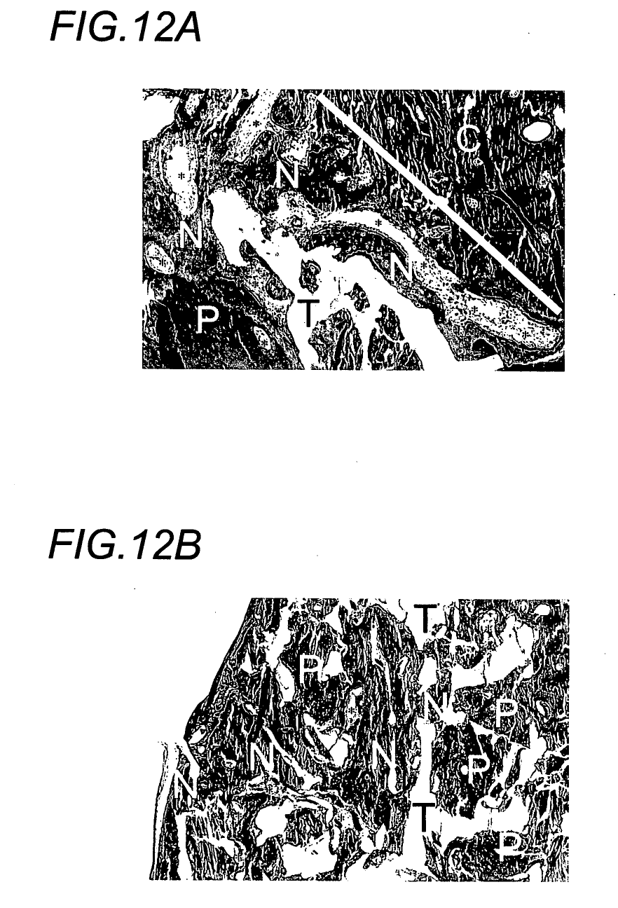

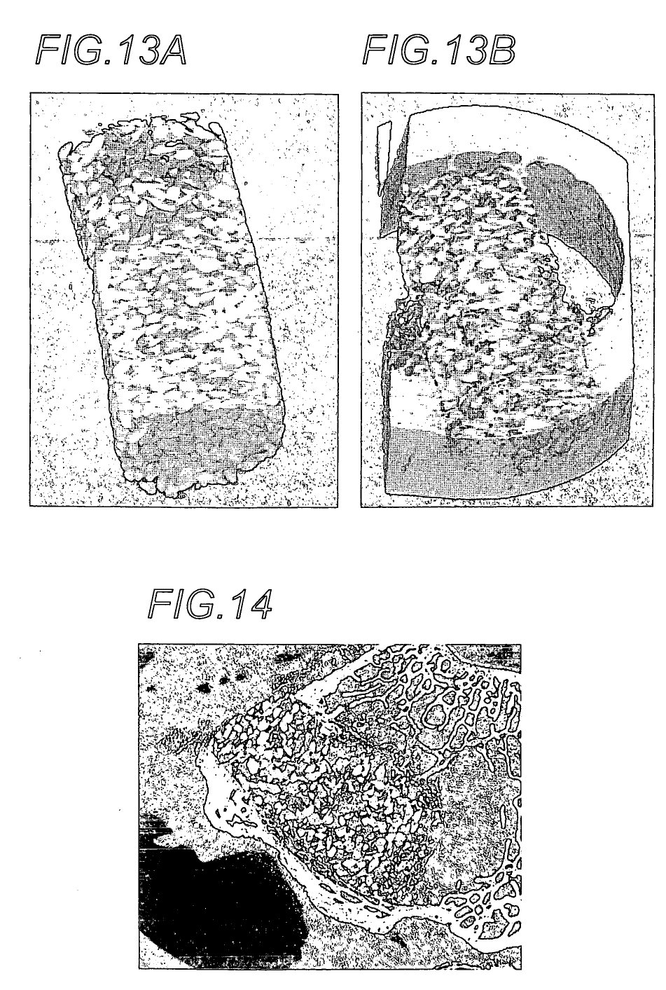

Pilot canine diaphyseal implantation histology from periods beginning at 28 days (FIG. 12) and ranging to 26 weeks indicates extensive healing and incorporation (FIG. 13). At all timepoints, cellular infiltration into the implants and bone formation on the allograft particles was substantial. New bone bridged from the defect border to the allograft particles, and within the implant from particle to particle, consistent with healing pattern of porous non weightbearing implants, (Ingram, R. et al. Ostoconductivity of tyrosine derived polycarbonate implants in condylar defects in 48th Annual Meeting of the Orthopaedic Research Society 737, Houston, Tex., 2002).

These results demonstrate that it is possible to achieve mechanical and biological properties similar to those of cortical bone from an allograft/biodegradable polymer composite. One of the composite formulations studied was a composite of 75% bone and 25% polyDTE-carbonate. When Ingram et al. evaluated a co-polymer from this polymer family in a rabbit condylar defect, it was shown to be osteoconductive, both for solid implants at the outer surfaces and for open pore foam constructs, where bony infiltration of the pores occurred by 6 weeks. Our data show that similar bony penetration of a weightbearing composite matrix can be achieved, using bone particles to reinforce the composite and to direct cell-mediated pore generation and ingrowth. The specific formulation, allograft particle size, polymer selection, and polymer to allograft ratio can dramatically influence the mechanical and biological performance.

### Example 3

Particles of dry cortical rabbit bone were mixed with polymer in a ratio of 60 allograft bone: 40 poly(lactide-co-glycolide) or 75 allograft bone: 25 polyDTE-carbonate by weight, packed into a Carver press, and pressed to 14,300 psi. The heat was then ramped to 110 degrees C. and the implant pressed again up to 14,300 psi. The finished shape of the molded implants was cylindrical, with a diameter of approximately 4.8 mm and length of approximately 15 mm. Implants were sterilized using conventional methods.

Each composite sample was inserted into a defect created in the lateral femoral condyle of male New Zealand White Rabbits. In each rabbit, a single implant was inserted in both the left and the right femoral condyle. A drill was used to create a hole in the lateral condyle of each femur. The hole was sized such as to allow insertion of the implant, but also provide a tight fit allowing good bone to implant contact. The leading edge of each implant was chamfered to aid insertion. After placement in the defect, the portion of the sample extending outside the site was ground off to be approximately flush with the surrounding bone. Stainless steel suture, cut to an appropriate length, was placed roughly parallel with the long axis of the implant, between the implant and the adjacent host bone. The section of suture was placed distally and slightly anterior to the implant to aid in detection and identify the orientation of the implant after healing.

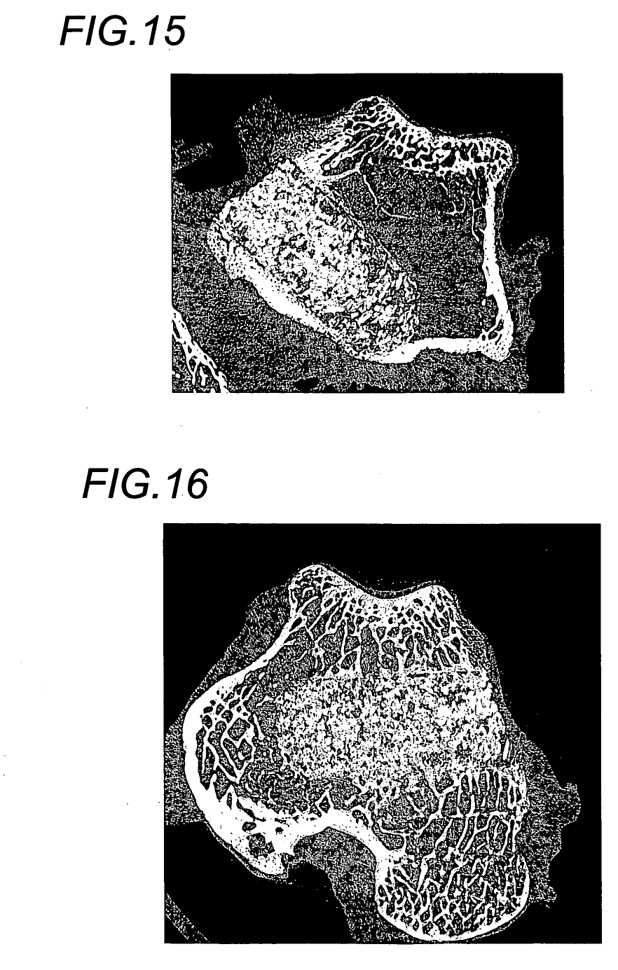

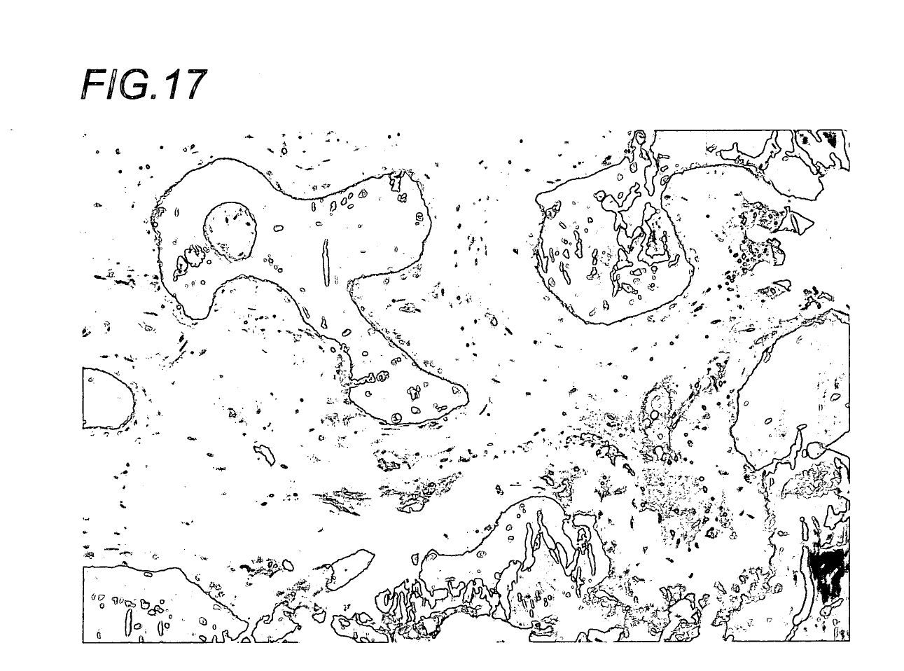

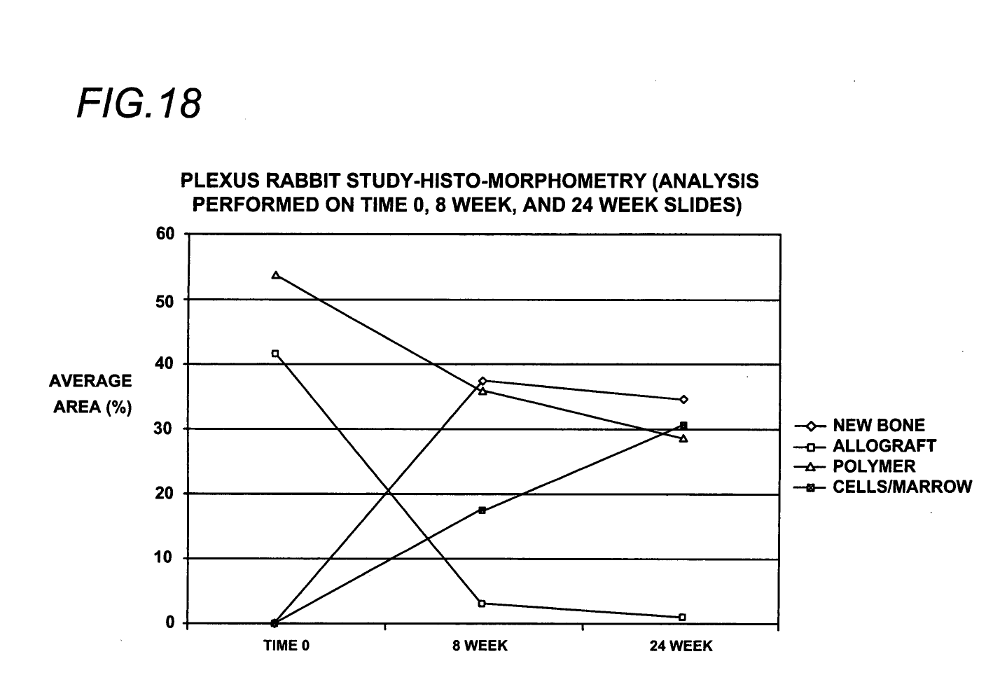

After four weeks, bone had already started to infiltrate the composites formed with both the lactide-glycolide co-polymer and polyDTE-carbonate (FIGS. 14, 15). Increased infiltration was observed when the composite was abraded with using a diamond abrasive wheel to expose the bone before implantation (FIG. 16). More complete infiltration of the composites is observed eight weeks post-implantation (FIG. 17). The replacement of allograft particles with new bone and infiltration of bone into the composite was quantified by marking the relevant regions of micrographs of tissue sections and using a computer to calculate the areas of each of the regions. The results for 75:25 composites using surface demineralized particles are shown in FIG. 18.

### Example 4

This example shows how varying the porosity of the material used as the cell conducting phase may be exploited to manipulate the mechanical properties of the composite.

In one embodiment, the cell conducting phase is a calcium phosphate material having porosity of 10 micrometers or less. If a sufficient quantity of a resorbable calcium phosphate is employed, it may provide a contiguous path for cells to penetrate into the composite. However, the contribution of the phase to the mechanical strength of the composite will be determined in part by surface interactions between the binder phase and the calcium phosphate material. These may be increased by creating chemical bonds between the cell conducting phase and the binder phase.

In a second embodiment, the cell conducting phase is a calcium phosphate material exhibiting porosity on the order of 150 micrometers. This porosity may become filled with the binder phase during fabrication of the composite, creating a mechanical interlock between the binder phase and the cell conducting phase and allowing the cell conducting phase to contribute to the mechanical strength of the composite without relying on chemical interactions with the binder phase. However, for the same dry volume of cell conducting particles, the weight fraction of calcium phosphate particles will be far less, and the volume of the composite occupied by calcium phosphate will also be less. This may limit the connectivity of the cell conducting phase and impede the penetration of tissue into the composite.

In a third embodiment, cortical bone is employed as the cell conducting phase. Cortical bone is mostly solid but includes pores left by cells and vascular tissue of about 100 micrometers. These pores detract from the conductivity of the phase but aid interpenetration of the binder phase with the cell conducting phase. As the cell conducting phase is transformed, tissue is introduced to the implant site that contributes to load bearing. The binder phase may continue to degrade at a slower rate, preventing wholesale degradation of the mechanical strength of material within the implant site and allowing the initial “ingrown” tissue to mature and remodel in response to physiological loading as the degrading material is replaced with new tissue.

These examples show that the microstructure of the cell conducting phase may be exploited to manipulate the mechanical strength of the implant and the mechanism of transformation of the implant. The load bearing ability of the implant depends on both the microstructure of the implant and the mechanical properties of its components. For example, cancellous bone may be employed as a cell conducting phase. When compared to cortical bone, a given weight fraction of cancellous bone will occupy a higher volume fraction of an implant. It may be desirable to use a higher weight fraction of cancellous bone to increase the connectivity of the cell conducting phase. Indeed, it may be possible to use higher weight fractions of cancellous bone than cortical bone because the interpenetration of the binder phase with the bone will increase the coherence of the composite.

The desired fraction of cancellous bone or other porous materials in the composite to provide a path for cells into an implant may still be determined experimentally. The cross-sections evaluated using micro CT should be thinner than the porosity of the cell conducting phase. Analysis of the connectivity of the porous material will then not only account for the connectivity of the cell conducting particles but of the material from which they are fabricated. Thus, the connectivity of the chemical composition of the cell conducting phase may be used as one factor in optimizing the physical microstructure of the composite.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

1. An implant comprising a cell conducting phase and a binder phase, wherein:
at least a portion of the surface of the implant comprises the cell conducting phase, and
the cell conducting phase defines a path from the surface of the implant to an interior of the implant.

2. The implant of claim 1, wherein at least a portion of the cell conducting phase, at least a portion of the binder phase, or both, swells upon exposure to a physiological environment.

3. The implant of claim 1, wherein the cell conducting phase comprises particles having a distribution of aspect ratios, and wherein the volume fraction of the cell conducting phase is at least as great as the percolation threshold of the implant for particles having an aspect ratio equal to the largest aspect ratio in the distribution.

4. The implant of claim 1, wherein the implant provides an environment that, in vivo, allows cells to penetrate at least 1 mm into the implant from the surface.

5. The implant of claim 1, wherein the implant provides an environment that, in vivo, allows tissue ingrowth to extend into the implant at least 1 mm from the surface.

6. The implant of claim 1, wherein the cell conducting phase comprises allograft bone.

7. The implant of claim 1, wherein the cell conducting phase exceeds about 50 weight percent of the implant.

8. The implant of claim 7, wherein the cell conducting phase exceeds about 60 weight percent of the implant.

9. The implant of claim 8, wherein the cell conducting phase exceeds about 70 weight percent of the implant.

10. The implant of claim 1, wherein the cell conducting phase exceeds about 27 percent by volume of the implant.

11. The implant of claim 10, wherein the cell conducting phase exceeds about 35 percent by volume of the implant.

12. The implant of claim 11, wherein the cell conducting phase exceeds about 40 percent by volume of the implant.

13. The implant of claim 12, wherein the cell conducting phase exceeds about 50 percent by volume of the implant.

14. The implant of claim 1, wherein at least a portion of the surface of the implant comprises a cell conducting material.

15. The implant of claim 1, wherein the cell conducting phase includes a connected cluster of cell conducting material that occupies at least 10% of the area of a cross section of the implant.

16. The implant of claim 1, wherein the cell conducting phase includes a connected cluster of cell conducting material that occupies at least 20% of the area of a cross section of the implant.

17. The implant of claim 1, wherein the cell conducting phase includes a connected cluster of cell conducting material that occupies at least 30% of the area of a cross section of the implant.

18. The implant of claim 1, wherein the cell conducting phase includes a connected cluster of cell conducting material that occupies at least 40% of the area of a cross section of the implant.

19. The implant of claim 1, wherein the cell conducting phase includes a connected cluster of cell conducting material that occupies at least 50% of the area of a cross section of the implant.

20. The implant of claim 1, wherein the cell conducting phase includes a connected cluster of cell conducting material that occupies at least 60% of the area of a cross section of the implant.

21. The implant of claim 1, wherein the cell conducting phase includes a connected cluster of cell conducting material that occupies at least 70% of the area of a cross section of the implant.

22. The implant of claim 1, wherein the cell conducting phase includes a connected cluster of cell conducting material that occupies at least 80% of the area of a cross section of the implant.

23. The implant of claim 1, wherein the cell conducting phase includes a connected cluster of cell conducting material that occupies at least 90% of the area of a cross section of the implant.

24. The implant of claim 1, wherein the cell conducting phase comprises a tissue-derived material having a plurality of pores with a size between 10 and 500 μm.

25. The implant of claim 1, wherein the implant lacks porosity sufficiently large to permit the migration of cells.

26. The implant of claim 1, wherein the ratio of the cell conducting phase to the binder phase exhibits a gradient proceeding from a portion of the surface of the implant to a predetermined portion of an interior of the implant.

27. The implant of claim 26, wherein the gradient is in the direction of decreasing cell conductor phase to binder phase ratio.

28. The implant of claim 1, wherein the cell conducting phase defines at least one blind path from a surface of the implant to a location in the interior of the implant.

29. The implant of claim 1, wherein the cell conducting phase, the binder phase, or both, include a member of a bioactive agent, biomolecule, or small molecule.

30. The implant of claim 29, wherein a concentration of the member exhibits a gradient between two predetermined locations in the implant.

31. The implant of claim 29, wherein a concentration of the member exhibits a gradient exhibiting radial symmetry.

32. The implant of claim 1, wherein the cell conducting phase comprises one or more of a tissue-derived material, an extracellular matrix component, a synthetic extracellular matrix analog, a polymer, and a ceramic material.

33. The implant of claim 32, wherein the cell conducting phase comprises a tyrosine-based polycarbonate, a polylactide, a polyurethane, or any combination of the above.

34. The implant of claim 32, wherein the cell conducting phase comprises a synthetic material.

35. The implant of claim 1, wherein the binder phase includes a cell conducting material.

36. The implant of claim 1, wherein the binder phase comprises one or more of a polymer and an inorganic material.

37. The implant of claim 1, wherein the implant exhibits a gradient in its transformation rate.

38. The implant of claim 1, wherein a predetermined portion of the implant is free of cell conducting material.

39. An implant comprising a cell conducting phase and a binder phase, wherein at least one cross-section of the implant exhibits a connected cluster of the cell conducting phase that defines a path from the surface of the implant to a location in the interior of the implant.

40-65. (canceled)

66. A composite material, comprising:
a cell conducting phase comprising bone fibers, wherein the long axis of the bone fibers corresponds to a long axis of a bone from which the bone fibers were derived; and
a binder phase combined with the cell conducting phase.

67. An implant disposed in an in vivo tissue site and comprising a composite, the composite comprising a cell conducting phase and a binder phase, wherein:
the cell conducting phase comprises a cell-free bone-derived material, and
at least one living cell derived from a host in which the implant is disposed is disposed within the implant.

68. The implant of claim 67, wherein at least one cell is disposed at least 1 mm from the surface of the implant.

69. An implant disposed in an in vivo tissue site and comprising a composite, the composite comprising a cell conducting phase and a binder phase, wherein:
the cell conducting phase comprises a cell-free bone-derived material, and
living tissue provides mechanical communication between an interior of the implant and tissue exterior to the implant.
